(19) United States
(12) Patent Application Publication
(10) Pub. No.: US 2023/0159702 A1
(43) Pub. Date: May 25, 2023

(54) METHOD OF PRODUCING A BIOACTIVE SURFACE

(71) Applicant: STICHTING RADBOUD UNIVERSITAIR MEDISCH CENTRUM, Nijmegen (NL)

(72) Inventors: Dion VOERMAN, Nijmegen (NL); Roelof HAMMINK, Nijmegen (NL); Martijn VERDOES, Nijmegen (NL); Carl Gustav FIGDOR, Nijmegen (NL)

(73) Assignee: STICHTING RADBOUD UNIVERSITAIR MEDISCH CENTRUM, Nijmegen (NL)

(21) Appl. No.: 17/432,278

(22) PCT Filed: Feb. 27, 2020
(86) PCT No.: PCT/EP2020/055132

§ 371 Date: Aug. 19, 2021

(30) Foreign Application Priority Data
Feb. 28, 2019 (GB) 1902702.8

(51) Int. Cl. C08G 69/08; C08B 37/08; G01N 33/543

(52) U.S. Cl. CPC C08G 69/08; C08B 37/0072; G01N 33/54353

(57) ABSTRACT

The present invention is directed to nanofilaments for polymer brushes, to polymer brushes comprising the nanofilaments and methods of making the same. In particular, the invention provides water soluble nanofilaments which may be grafted to a surface and which may be functionalised with (bio)molecules.

### FIELD OF INVENTION

The present invention is directed to nanofilaments for polymer brushes, to polymer brushes comprising the nanofilaments and methods of making the same. In particular, the invention provides water soluble nanofilaments which may be grafted to a surface and which may be functionalised with (bio)molecules.

### BACKGROUND

Surfaces that interact specifically with biological systems are crucial for many applications, including tissue engineering, bio-sensing, medical implants and nanomedicine. The bulk materials used for these applications often lack the properties needed for the desired biological interactions and therefore bioactive surface modifications are applied. Biomolecules are often used as functional groups on these bioactive surfaces and can be attached to a surface via physical adsorption, ligand-receptor interactions such as the biotin streptavidin pair or via covalent attachment. For example, growth factors can be attached to a surface to promote proliferation, antimicrobial peptides for bacterial killing and RGD peptide motifs may be used to increase cell adhesion. The choice of functional groups is of course an important design parameter, but also the physical and chemical architecture and properties of a surface coating are important in governing the interactions with the biological systems.

Examples of such properties are hydrophobicity, surface charge, stiffness and surface roughness and patterning. Hydrophobicity is well known to dictate non-specific binding which in turn directly influences cell signalling. Surface stiffness can tune the outcome of surface interactions with cells, whilst surface roughness and patterning can modify (bio)fouling and cell interactions. In addition, the manner in which the functional groups are attached to the surface can be a determining factor in their function.

Polymer brush surfaces have proven to be a platform which allows for control of these interactions. In a polymer brush, polymers are tethered at one end to a surface at grafting densities high enough to crowd the polymers and force them to extend in the direction perpendicular to the surface due to entropic expulsion. In comparison with other surface coating techniques (e.g. self-assembled monolayers or spincoating of polymers), polymer brushes provide chemical and mechanical stability as well as control over layer thickness (tunable by polymer length and density) and composition (tunable by choice of monomer and polymer).

Brush surfaces can be prepared via ‘grafting to’ or ‘grafting from’ strategies. With ‘grafting to’ strategies, pre-formed polymers are tethered by one end to a surface. Alternatively, ‘grafting from’ strategies grow the polymer chains from a surface. The obtained densities of brushes prepared by ‘grafting to’ strategies are often limited by steric hindrance, meaning ‘grafting from’ is often preferred. Most commonly, controlled radical polymerizations are used, such as atom transfer radical polymerization (ATRP), reversible radical addition-fragmentation chain transfer polymerization (RAFT) or nitroxide mediated polymerization (NMP). Also, for non-water soluble PIC polymers made from isocyano-D-alanine-L-alanine-methoxy monomers, polymer brushes produced by surface initiated nickel polymerization have been described (Lim etal. (2008) Macromolecules 41, 1945-1951, which is incorporated herein by reference).

A range of different polymers have already been used to synthesize polymer brushes, including polyethylene glycol (PEG), polisocyanopeptide (PIC), poly-acrylamides such as N-isopropyl acrylamide (NIPAM) and acrylates such as poly(ethylene glycol)methacrylate (POEGMA) .

Unfortunately, for some classes of polymers, the choice of solvent can affect the length of polymer chains achievable in “grafting from” approaches. This can restrict the choice of surface used due to a restriction on the solvents used for polymerisation.

Accordingly, there remains a need for improved, or at least alternative, nanofilaments for use in ‘grafting to’ approaches which can achieve high density and useful polymer lengths as well as methods of producing the same.

### SUMMARY OF INVENTION

As demonstrated herein, the problem of providing nanofilaments which can be used in “grafting to” approaches to producing polymer brushes is solved by providing nanofilaments with a first portion which includes functional groups suitable for attachment to a surface, and second portion with a functional group that may be used to decorate the filaments with molecules, for example biomolecules.

Nanofilaments according to the invention and those produced according to the methods of the invention are advantageous as they can be attached to a surface, for example to form a polymer brush.

As demonstrated herein, the block copolymer structure of the nanofilaments according to the invention provides multiple binding sites for attachment to a surface. Advantageously, this provides significantly better levels of specific attachment compared to nanofilaments with only a single binding site for attachment.

In addition, because they comprise two portions each with different functional groups, the range of options for reactions to attach nanofilaments of the invention to biomolecules or to surfaces is expanded. For instance, biomolecules can be attached first under reaction conditions that would not be compatible with the intended surface. The nanofilament with biomolecule attached can then be attached to the surface under surface-compatible reaction conditions. This advantageously expands the range of surface materials and biomolecules to which the nanofilaments can be attached, as well as the range of functional groups suitable to be used for each attachment.

Furthermore, the nanofilaments and methods for their production according to the invention advantageously enable attachment to a surface of nanofilaments which require polymerisation in a solvent that is not compatible with the surface material to which the nanofilaments are to be attached. This is achieved by having two different functional groups that remain stable under polymerisation conditions - that is, are still functional in the produced nanofilaments. Once produced, the first functional group of the nanofilaments can be selectively reacted such that it can be attached to a surface, leaving the second functional group free to be decorated with a biomolecule, for example.

The advantages of the present invention are evident from the Examples. By way of examples of the invention, production of PIC nanofilaments according to the invention is described, plus their specific attachment to a variety of surfaces. Until the present invention, it was not possible to use PIC nanofilaments in a “grafting to” approach. In addition, it has not previously been possible to form polymer brushes from water-soluble PIC nanofilaments. This has limited the surfaces to which the PIC nanofilaments can be attached. The block copolymer nanofilaments of the present invention make this possible for PIC nanofilaments. Similar advantages will be achievable for nanofilaments based on other polymers sharing similar properties to PIC.

Accordingly, in a first aspect there is provided a nanofilament for forming a polymer brush, wherein the nanofilament comprises first and second portions;
wherein the first portion comprises a plurality of functional repeat units each having a first functional group and optionally a plurality of non-functional repeat units;
wherein the second portion comprises a plurality of functional repeat units each having a second functional group and optionally a plurality of non-functional repeat units; and
wherein the first and second functional groups are different.

In a further aspect is provided a method of producing a nanofilament for a polymer brush, the method comprising either:
(a)
   (i) providing a first polymerisable composition comprising an amount of a first monomer having a first functional group and an amount of a catalyst and optionally an amount of a second non-functional monomer;
   (ii) polymerizing the first monomer, and if present the non-functional monomer, to provide a first polymer composition;
   (iii) adding an amount of a second monomer having a second functional group, and optionally an amount of a non-functional monomer, to the first polymer composition to provide a second polymerisable composition; and
   (iv) polymerizing the second monomer, and if present the non-functional monomer, to produce the nanofilament; or
(b)
   (i) providing a first polymerisable composition comprising an amount of a second monomer having a second functional group and an amount of a catalyst and optionally an amount of a non-functional monomer;
   (ii) polymerizing the first monomer, and if present the non-functional monomer, to provide a first polymer composition;
   (iii) adding an amount of a first monomer having a first functional group, and optionally an amount of a non-functional monomer, to the first polymer composition to provide a second polymerisable composition; and
   (iv) polymerizing the first monomer, and if present non-functional monomer, to produce the nanofilament;
wherein the first and second functional groups are different.

In a further aspect is provided a nanofilament obtainable by a method according to the invention.

In a further aspect is provided a polymer brush comprising a plurality of nanofilaments according to the invention.

In a further aspect is provided a method of producing a polymer brush comprising binding to a surface a plurality of nanofilaments according to the invention.

In a further aspect is provided a scaffold comprising a surface to which a plurality of nanofilaments according to the invention has been bound.

In a preferred embodiment of all aspects of the invention, the nanofilament is a PIC nanofilament.

The present disclosure will now be described further. In the following passages different aspects/embodiments of the disclosure are defined in more detail. Each aspect/embodiment so defined may be combined with any other aspect/embodiment or aspects/embodiments unless clearly indicated to the contrary. In particular, any feature indicated as being preferred or advantageous may be combined with any other feature or features indicated as being preferred or advantageous.

### FIGURES

FIG. 1: Structures of the monomer and polymers used to test binding to 1 µm beads and binding results. a) structure of methoxy monomer 1 and azide monomer 2. b) Structure of PIC1a-c c) Structure of PIC2-4. d) Binding of azide functional PIC to DBCO beads. e) Binding of biotin functional PIC to streptavidin beads. Mean +/- standard deviation of three independent experiments is shown.

FIG. 2: Design of the PIC with new allyl monomers in the first block that can be further derivatized with the NITEC reaction. a) Structure of the allyl monomer, b) Reaction mechanism of the NITEC reaction. The fluorescent pyrazoline is depicted in red. c) General structure of PIC5a-e. The part in red depicts the firs block containing allyl functionality. d) STORM images of PIC5b that was labeled with AlexaFluor 647 (yellow) in the second block and AlexaFluor 488 (blue) in the first block. Each five pixel cross represents the localization of a single dye with an accuracy of 12 nm (AlexaFluor 647) or 20 nm (AlexaFluor 488). The scale bar represents 50 nm.

FIG. 3: Investigation of biotin conjugation of PIC5a-f by measuring fluorescence of the NITEC reaction product and subsequent binding biotinylated polymers to streptavidin beads. a) Schematic representation of the two-step binding protocol of the allyl polymers using tetrazole-PEG₈-biotin. b) Fluorescence of conjugated biotin of PIC5a-f when reacted with an excess of tetrazole-biotin (200 eq per polymer). c) Binding of the biotinylated PIC5a-f to streptavidin beads after labeling with AlexaFluor647 as measured by mean fluorescence intensity on beads by flow cytometry. d) Fluorescence of photoclick of PIC5b when reacted with different equivalents of tetrazole-biotin per allyl. e) Binding of the biotinylated PIC5b from graph b to streptavidin beads after labeling with AlexaFluor647 as measured by mean fluorescence intensity on beads by flow cytometry. Values plotted are means +/- standard deviation of three independent experiments.

FIG. 4: Binding of different amount of labeled biotinylated PIC5b to streptavidin microbeads. a) MFI signal on the beads versus amount of polymer added, b) µg polymer on beads as determined by the stripping assay plotted against amount of polymer added. c) Correlation between MFI signal and calculated polymer density. Mean +/- standard deviation of three independent experiments is shown.

FIG. 5: Mean fluorescence intensity (MFI) of Atto 488 labeled BSA and Alexafluor 647 labeled PIC on microbeads and nanoparticles. a) MFl’s for the different conditions used for binding to the streptavidin microbeads. b) MFl’s for the different conditions used for binding to the streptavidin nanoparticles. Mean +/- standard deviation of three independent experiments is shown.

FIG. 6: Activation of T cells with either PIC-Ab beads (square) or only Ab beads (circle). (a) and (b) show the effect of the ratio of CD3/CD28 antibodies on the beads on T cell cytokine production; (c) and (d) show the effect of antibody density per bead on cytokine production, and (e) shows the percentage of cytokine producing cells induced by PIC-Ab beads compared to Ab-only beads. (f) shows T cell proliferation as measured by mean division cycle after 3 days. Antibody density effects were compared at a 4:1 ratio of aCD28:aCD3. IFNy and IL-2 production was measured after 24h.

FIG. 7: T cell cytokine production following 24 hours of incubation with PIC-Ab beads (aCD3:aCD28). “Flat PlC″ - cells exposed to beads to which the PIC filaments are attached in a “flat” configuration - i.e. along their length such that they do not form a polymer brush; “PIC brush” - cells exposed to beads to which the PIC filaments are attached so as to form a polymer brush; “Crosslinked PlC” - cells exposed to beads to which the PIC filaments are attached so as to form a polymer brush, but where the filaments are subsequently cross-linked. IFNy and IL-2 production was measured after 24h.

FIG. 8: Comparison of PIC-Ab beads with commercial CD3/CD28 Dynabeads® and Miltenyi Transact®. (A) T cell IFNy and (B) IL-2 production measured after 24h incubation; (C) T cell proliferation as measured by mean division cycle after 3 days; (D) Fold expansion of cells after 7 and 14 days of incubation.

FIG. 9: Comparison of PIC-Ab beads with commercial CD3/CD28 Dynabeads® and Miltenyi Transact®.

FIG. 10: T cell proliferation induced by aCD3:aCD28 antibody-functionalised nanofilaments formed of polymers of alginate or dextran. T cell proliferation as measured by mean division cycle after 3 days.

FIG. 11: Synthetic scheme for hyaluronic acid-poly-glutamate block co-polymer.

### DETAILED DESCRIPTION

The present invention relates to nanofilaments for forming polymer brushes, and methods for producing said nanofilaments. Nanofilaments according to the invention are substantially linear polymers having functional groups for binding to (bio)molecules and which can be bound to a surface. Nanofilaments for polymer brushes are known, as discussed above. However, they do not have the structure described herein. In particular, the inventors have developed specific nanofilaments which may be ‘grafted to’ a surface with a sufficient binding density to provide a useful polymer brush.

The term polymer brush is well-known in the art, and refers to a surface decorated with polymers at grafting densities high enough to crowd the polymers and force them to extend in a direction substantially perpendicular to the surface due to entropic expulsion.

The term “polymer,” as used herein, refers to a polymeric compound prepared by polymerizing monomers, whether of the same or a different type. The generic term polymer thus encompasses the term homopolymer (employed to refer to polymers prepared from only one type of monomer, with the understanding that trace amounts of impurities can be incorporated into the polymer structure), copolymer and interpolymer. The terms “interpolymer” and “copolymer” are used interchangeably to refer to polymers prepared by the polymerization of at least two different types of monomers.

### Nanofilaments

According to a first aspect of the invention there is provided a nanofilament for forming a polymer brush, wherein the nanofilament comprises first and second portions;
wherein the first portion comprises a plurality of functional repeat units each having a first functional group and optionally a plurality of non-functional repeat units;
wherein the second portion comprises a plurality of functional repeat units each having a second functional group and optionally a plurality of non-functional repeat units; and
wherein the first and second functional groups are different.

The nanofilament comprises a first portion comprising a plurality of functional repeat units each having a first functional group and optionally a plurality of non-functional repeat units. The first portion of the nanofilament is at a first end of the nanofilament and is for binding the nanofilament to a surface. Accordingly, the first functional group is for binding to a surface to be functionalised with a nanofilament.

The nanofilament of the first aspect further comprises a second portion comprising a plurality of functional repeat units each having a second functional group and optionally a plurality of non-functional repeat units. The second portion preferably makes up the majority of the nanofilament. The second portion is intended to be ‘decorated’ with molecules such as biomolecules to provide the desired functionalisation. Accordingly, the second functional group is for binding to molecules to be bound to the nanofilaments.

By functional repeat unit it is meant that the repeat unit includes a functional group suitable for binding to another entity such as a surface or molecule. That is, a functional repeat unit is a repeat unit having a functional moiety/functional group suitable for use in an attachment reaction, such as a so-called click reaction. Suitable functional moieties will be familiar to the skilled person and include those commonly used in so-called click chemistry, such as those described herein.

As already described, the first and second functional groups serve different purposes in the nanofilament. The functional repeat units of the first portion are for grafting the nanofilament to a surface to form a polymer brush, whereas the functional repeat units of the second portion are for binding to molecules to provide the desired functionalisation. The first and second functional groups are therefore different so that they can be selectively reacted to achieve the desired binding.

For example a nanofilament of the present invention may be bound to a surface using reaction conditions under which the first functional group reacts and the second functional group does not react. A nanofilament of the invention may be decorated with biomolecules using reaction conditions under which the first functional group does not react but the second functional group does react.

Alternatively, the first and second functional groups may be based on the same functional moiety except that either the first or second functional group is protected by a protecting group thereby preventing it from reacting until the protecting group is removed. For example the first functional group may be an ester and the second functional group may be a carboxylic acid. The carboxylic acid may be reacted to decorate the nanofilament with the desired molecules then the ester may be converted to the underlying acid and reacted to bind the nanofilament to a surface.

The above are provided as examples and the skilled person would readily appreciate that the choice of the first and second functional group could readily be reversed without affecting the working of the invention.

Preferably the first and second functional groups are not different by virtue of the presence of a protecting group but are distinct chemical groups.

Preferably the first and second functional groups are different groups selected from azide, N-hydroxysuccinimide (NHS), vinyl sulfone, allyl, alkynyl (e.g. acetylene, DBCO and BCN), NH₂, COOH, maleimide, tetrazine, transcyclooctene (TCO), thiol and alkoxy. These groups have shown utility in binding to surfaces and molecules to be attached to the polymer by known methods such as so-called click chemistry.

In a preferred embodiment, the first functional group is azide. Alternatively, preferably, the first functional group is allyl.

In a preferred embodiment, the second functional group is azide. Alternatively, preferably, the second functional group is allyl.

By non-functional repeat unit it is meant that the repeat unit does not include such a functional moiety/ functional group. Primarily, the non-functional repeat units serve as spacers to separate the functional repeat units and increase the overall length of the nanofilament without including excess functional moieties.

The non-functional repeat units in the first and second portions may be the same or different. In some embodiments the non-functional repeat units are the same in both the first and second portions. Advantageously this may provide benefits in respect of synthetic simplicity.

In some preferred embodiments the second portion may further comprise a plurality of functional repeat units comprising a third functional group, wherein the third functional group is different to the first and second functional groups. The above teaching regarding the first and second functional groups applies equally to the third functional group where present. Advantageously the inclusion of a plurality of further functional repeat units in the second portion may allow the selective binding of different molecules to the nanofilament if desired.

Preferably the nanofilament of the present invention is water soluble.

Solubility may be affected by the temperature at which it is measured. As would be understood by the skilled person, the term water-soluble in relation to the nanofilaments of the invention means that the polymer is sufficiently soluble in water that the polymer can be bound to a surface or can be decorated with (bio)molecules in water or an aqueous solution. In preferred embodiments, the nanofilaments of the invention are also water soluble at biological temperatures, for example around 37° C.

In certain embodiments, the nanofilament is formed of a polysaccharide polymer or co-polymer. Polysaccharide polymer-based nanofilaments may be formed of a polymer including those comprising one or more polysaccharides selected from: hyaluronic acid, alginate, pectin, chitin, guar, carrageenan, carboxymethylcellulose, heparin, and dextran.

In certain embodiments, the first and/or second portion of the nanofilament comprises a polysaccharide polymer.

In certain embodiments are provided alginate polymer nanofilaments. In certain embodiments are provided dextran polymer nanofilaments.

In certain embodiments are provided nanofilaments formed of a polysaccharide-glutamate co-polymer, wherein the first portion of the nanofilament comprises a poly-glutamate polymer and the second portion of the nanofilament comprises a polysaccharide polymer. The polysaccharide may be any one of hyaluronic acid, alginate, pectin, chitin, guar, carrageenan, carboxymethylcellulose, heparin, and dextran. In certain such embodiments, the nanofilament may be formed of a block co-polymer that is a hyaluronic acid-glutamate co-polymer. A suitable synthesis strategy for polysaccharide-poly-glutamate block co-polymer nanofilaments according to the invention is provided in the Examples, with reference to a hyaluronic acid-glutamate block co-polymer.

In a preferred embodiment the nanofilament is a polyisocyanopeptide (PIC).

Recently, water-soluble polyisocyanopeptides (PIC) have emerged as a new class of synthetic polymers with promising characteristics for interaction with cells. Advantageously these polymers have a higher than normal stiffness, that resembles more closely that of natural protein based filaments, which arises from their helical backbone that is stabilized by hydrogen bonding between the side chains. PIC nanofilaments decorated with antibodies and cytokines have shown to be very effective as a scaffold to present biological signals to cells (Mandal et al. ACS Chem. Biol. 2014; Mandal et al. Chem. Sci.(2013) 4, 4168; and GB1801902.6, each of which is incorporated herein by reference). The observed effective signalling is thought to arise from the polymers’ semi-flexible backbone in combination with their length. The stiffer PIC backbone prevents the polymer from collapsing. Instead it adopts a stretched out conformations allowing multiple bioactive groups to be positioned over a range of several hundreds of nanometers. The polymer is, however, flexible enough to allow for optimum multivalent binding of the targeted molecules. The combination of their length, stiffness and easy functionalization makes PICs suitable candidates for the development of bioactive polymer brush surfaces

Suitable PIC nanofilaments may be synthesised according to methods known in the art by polymerisation of isocyanopeptide monomers (see for example Mandal, S et al. Therapeutic Nanoworms: Towards Novel Synthetic Dendritic Cells for Immunotherapy. Chem. Sci. 2013, 4, 4168; and Koepf et al, European Polymer Journal, 49 (6), 2013 p.1510-1522, each of which are incorporated herein by reference).

Suitable methods for preparation of PIC polymer backbones are also described in WO2011007012 and GB1801902.6, each of which is incorporated herein by reference for this purpose.

In certain embodiments, the PIC nanofilaments can be prepared from isocyanide monomers that have been derived from amino acids or peptides. Techniques for converting amino acids into isocyanopeptides suitable for use as monomers in the PIC polymers are known in the art.

When an isocyanopeptide monomer is derived from an amino acid, it may be derived from any suitable naturally-occurring or non-naturally-occurring amino acid, and may for example be a D- or L- amino acid (or may have an R-confirmation or an S-confirmation, referring to the chiral alpha carbon). Selection of a suitable isomer would be familiar to the skilled person and may be determined, for example, by the intended “handedness” of the PIC polymer, which is helical in structure.

Although PICs have been found to be particularly useful, as discussed above, their use has been limited due to difficulties in producing polymer brushes comprising PICs.

While ‘grafting from’ strategies have been successfully employed to create a wide variety of different bioactive surfaces, a downside is that they often require organic solvents during polymerization. The requirement of organic solvents limits the scope of surfaces that can be functionalized as some surface materials are only stable under aqueous conditions. Examples include many plastics used for cell culturing such as poly(methyl methacrylate) (PMMA) or polystyrene (PS). Also nano- or microparticles made from poly-lactic-glycolic acid (PLGA) or cell surfaces are not resistant to organic solvents but can benefit from surface modifications. To extend the use of bioactive surfaces to these materials, synthesis under aqueous conditions is required.

For the PIC, however, it has been found that organic solvents are necessary to obtain polymers of the desired length. Advantageously, the present invention may provide a water soluble PIC nanofilament which may be functionalized and grafted to a surface.

In preferred embodiments of all nanofilaments of the invention, the functional group of the first repeat unit is selected from azide, N-hydroxysuccinimide (NHS), vinyl sulfone, allyl, alkynyl (e.g. acetylene, DBCO and BCN), NH₂, COOH, maleimide, tetrazine, transcyclooctene (TCO), thiol and alkoxy.

In preferred embodiments, the functional group of the second repeat unit is selected from azide, N-hydroxysuccinimide (NHS), vinyl sulfone, allyl, alkynyl (e.g. acetylene, DBCO and BCN), NH₂, COOH, maleimide, tetrazine, transcyclooctene (TCO), thiol and alkoxy.

In a preferred embodiment, the first functional group is azide. Alternatively, preferably, the first functional group is allyl.

In a preferred embodiment, the second functional group is azide. Alternatively, preferably, the second functional group is allyl.

In certain preferred embodiments, the first or second functional repeat units each comprise an azide functional group. It is particularly preferred that the second functional group is azide as it means (bio)molecules can be easily conjugated to the nanofilament using the strain promoted azide-alkyne cyclo-addition (SPAAC) reaction.

In certain preferred embodiments, the first or second functional repeat units each comprise an allyl functional group. It is particularly preferred that the first functional group is an allyl group because the allyl is small, easy to introduce into the monomer and shows excellent reactivity with tetrazoles in the nitrile imine-mediated tetrazole-ene cycloaddition (NITEC), allowing for binding to surfaces and molecules even under biological conditions.

In certain preferred embodiments:
(a) the first functional group is allyl and the second functional group is azide, or
(b) the first functional group is azide and the second functional group is allyl.

In certain embodiments the first and/or second functional group is attached the respective functional repeat units via a spacer. A spacer is a moiety covalently attaching the functional group to the backbone of the nanofilament. Advantageously, conjugation of a functional group via a spacer can provide flexibility that may be tuned for optimizing functional interactions.

In certain preferred embodiments where the first functional repeat unit comprises a spacer, the spacer is a PEG spacer. In certain such embodiments, the PEG spacer comprises from 1-10 ethylene glycol units. In certain preferred embodiments, the PEG spacer comprises from 1-5 ethylene glycol units. In certain preferred embodiments, the PEG spacer comprises 1, 2, 3, 4 or 5 ethylene glycol units. In certain preferred embodiments, the PEG spacer comprises 3 ethylene glycol units.

In certain preferred embodiments where the second functional repeat unit comprises a spacer, the spacer is a PEG spacer. In certain such embodiments, the PEG spacer comprises from 1-10 ethylene glycol units. In certain preferred embodiments, the PEG spacer comprises from 1-5 ethylene glycol units. In certain preferred embodiments, the PEG spacer comprises 1, 2, 3, 4 or 5 ethylene glycol units. In certain preferred embodiments, the PEG spacer comprises 3 ethylene glycol units.

In certain embodiments the nanofilament comprises functional repeat units of the formula:

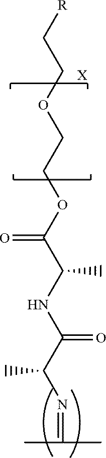

wherein R is the functional group capable of attaching the nanofilament to a molecule or a surface, and
wherein x is 1 to 10, preferably x is 3-10. In a preferred embodiment, x is 3.

In certain embodiments, the first portion comprises a plurality of non-functional repeat units. In such embodiments, the non-functional repeat units do not possess a reactive end group. In certain such embodiments, the non-functional repeat units comprise a methoxy end group.

In certain embodiments, the second portion comprises a plurality of non-functional repeat units. In such embodiments, the non-functional repeat units do not possess a reactive end group. In certain such embodiments, the non-functional repeat units comprise a methoxy end group.

In those embodiments where either or both the first and second portion comprises a non-functional repeat unit, the non-functional repeat units preferably have the formula:

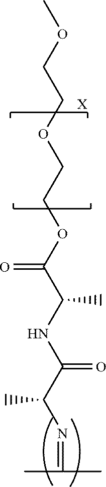

wherein x is 1 to 10, preferably x is 3-10. In a preferred embodiment, x is 3.

In certain embodiments wherein the nanofilament is not a PIC nanofilament, the nanofilament is a polysaccharide-poly-glutamate copolymer (e.g. a hyaluronic acid-poly-glutamate copolymer). In certain such embodiments, the nanofilament comprises first functional repeat units of the formula:

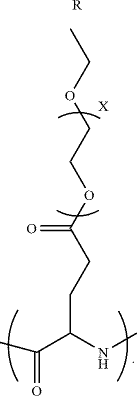

wherein R is the functional group capable of attaching the nanofilament to a molecule or a surface, and
wherein x is 1 to 10, preferably x is 3-10. In a preferred embodiment, x is 4. In a preferred embodiment, R is an allyl group.

In certain such embodiments, the nanofilament is a hyaluronic acid-poly-glutamate nanofilament and comprises second functional repeat units comprising the structure:

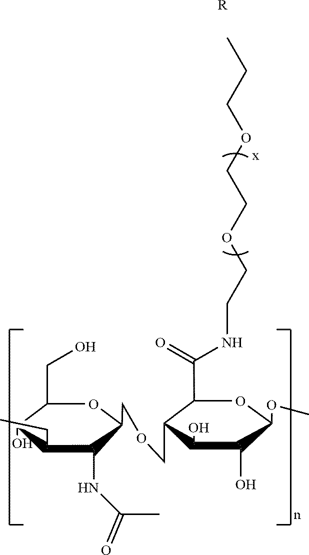

wherein R is the functional group capable of attaching the nanofilament to a molecule or a surface, and
wherein x is 1 to 10, preferably x is 3-10. In a preferred embodiment, x is 4. In a preferred embodiment, R is an azide group.

In those embodiments where the first portion comprises a non-functional repeat unit, the non-functional repeat units preferably have the formula:

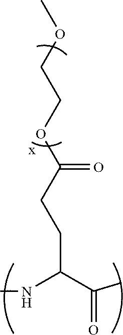

wherein x is 1 to 10, preferably x is 3-10. In a preferred embodiment, x is 4.

It has been found that when the nanofilament comprises a PEG spacer, the length of the PEG spacer may alter the water solubility of the polymer/nanofilament. Accordingly, for water soluble nanofilaments longer PEG spacers may be preferred, for example PEG spacers comprising at least 3 glycol units. In preferred embodiments, the PEG spacer comprises from 3-10 glycol units, preferably 3-5 glycol units, more preferably 3 or 4 glycol units, most preferably 3 glycol units. These preferred embodiments apply equally and independently to the functional and non-functional repeat units.

It is demonstrated herein that multiple functional groups in the first portion of the nanofilaments enhances the grafting to a surface. In particular, it is shown that filaments of the present invention show improved binding to surfaces due to the number and density of the functional groups in the first portion of the nanofilament.

Thus, in certain embodiments where the first portion comprises functional and non-functional repeat units, the ratio of non-functional to functional repeat units in the first portion is from 50:1 to 1:20. In certain embodiments, the ratio of non-functional to functional repeat units in the first portion is from from 30:1 to 1:10, optionally 20:1 to 1:10, optionally 10:1 to 1:10. In certain preferred embodiments the ratio of non-functional to functional repeat units in the first portion is from 1:1 to 1:10. In certain preferred embodiments, the ratio of non-functional to functional repeat units is selected from 1:1, 1:3, 1:5, 1:9 and 1:10.

Preferably the ratio of non-functional to functional repeat units in the second portion is from 100:1 to 10:1, preferably from 50:1 to 10:1, preferably from 35:1 to 25:1. In preferred embodiments, the ratio of non-functional to functional repeat units in the second portion is 30:1.

Preferably both the first and second portions of the nanofilament comprise a random arrangement of functional and non-functional repeat units. That is, preferably the two portions may be random copolymers.

Preferably, the polymer chain of the nanofilament according to the invention has a total length that is suitable for use in polymer brushes.

Preferably the nanofilament comprises from 1,000 to 20,000 repeat units, preferably from 1,000 to 10,000, preferably 1,500 to 5,000.

In certain embodiments, the backbone of the nanofilament is less than 1 micrometre in length. In certain embodiments, the backbone has a length in the range of from 1 nm to 1000 nm. In certain embodiments, the backbone has a length in the range of from about 5 nm to about 800 nm. In preferred embodiments the backbone has a length of from about 50 nm to about 800 nm, about 100 nm to about 700 nm, for example about 100 nm to about 500 nm, e.g. about 200 nm, about 300 nm, about 400 nm or about 500 nm. In certain preferred embodiments, the nanofilament has a length of about 200 nm. In certain preferred embodiments, the nanofilament has a length of about 400 nm.

It will be appreciated by the skilled person that, when synthesising a polymer or co-polymer, a statistical distribution of nanofilament lengths is obtained. The embodiments of length described above therefore refer to the statistical length of the polymer nanofilament sample, for example the mean length of polymer nanofilament.

Preferably the first portion comprises from 2 to 200 repeat units, preferably from 50 to 150 repeat units. Preferably the first portion comprises 100 repeat units or 50 repeat units.

### Nanofilaments Comprising Biomolecules

In certain embodiments, nanofilaments in accordance with the invention may be decorated with a plurality of a first biomolecule. Such biomolecules may be attached to the nanofilament via the functional repeat units of the second portion.

In such embodiments, biomolecules will be attached to the functional repeat units of the second portion as a result of reaction with the functional group present on the repeat units. It will be appreciated that nanofilaments decorated with biomolecules as a result of such an attachment reaction are still nanofilaments of the invention, albeit where the second functional group has undergone a reaction.

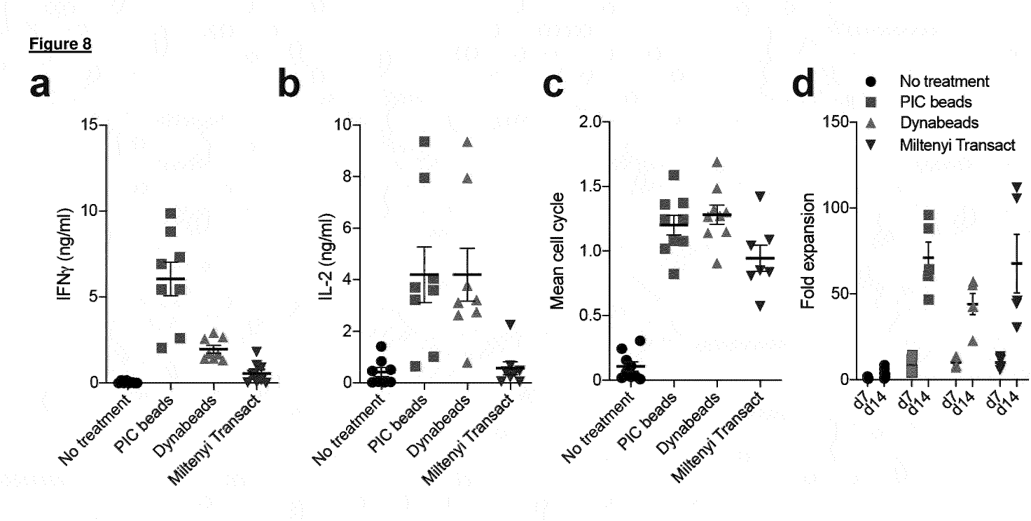

As demonstrated herein, nanofilaments decorated with biomolecules are particularly advantageous because surfaces functionalised with biomolecules attached to nanofilaments according to the invention are more potent and effective than surfaces where the biomolecules are attached directly (see FIG. 7). Nanofilaments decorated with biomolecules and their effects on target cells are described in WO2012/004369, Mandal et al., Chem. Sci., 2013,4, 4168-4174; Mandal et al ACS Chem Biol. 2015 Feb 20;10(2):485-92; Hammink et al ACS Omega 2017, 2, 937-945; Eggermont LJ et al. Advanced Therapeutics, 1(6), 2018, 1800021; and PCT/EP2019/052922, each of which is incorporated herein by reference. The biomolecule-decorated nanofilaments described in these documents will also be advantageously adapted for attachment to surfaces in accordance with the present invention.

Without wishing to be bound by theory, the improved efficacy of nanofilaments according to the invention for functionalising surfaces with biomolecules is thought to be due to improved presentation, particularly when the nanofilaments form a polymer brush on the surface. PIC nanofilaments provided herein are further advantageous due to the semi-flexible nature of PIC nanofilaments providing a degree of freedom sufficient to improve the fidelity and number of contacts of the biomolecule with a target cell receptor or ligand.

Thus, in certain embodiments there is provided a nanofilament for forming a polymer brush, wherein the nanofilament comprises first and second portions;
wherein the first portion comprises a plurality of functional repeat units each having a first functional group and optionally a plurality of non-functional repeat units;
wherein the second portion comprises a plurality of functional repeat units and optionally a plurality of non-functional repeat units; and
wherein the second portion further comprises a plurality of a first biomolecule, each biomolecule attached to a functional repeat unit of the second portion.

Biomolecules are biologically active molecules, for example growth factors, cytokines, haptens, cell surface receptors (e.g. MHC complexes, T cell receptors), co-stimulatory factors (e.g. CD28, CD3), inhibitory factors (e.g. PD-1, PD-L1, CTLA4, TIM3, TIGIT), integrins, selectins, their associated natural binding partners and antibodies which specifically bind to any of the foregoing biomolecules.

Where a nanofilament as provided herein comprises a plurality of a first biomolecule, it is decorated with 2 or more molecules of the biomolecule in question. Having multiple biomolecules attached to a PIC nanofilament is particularly beneficial as it takes advantage of the semi-flexible nature of the nanofilament. The semi-flexible nature allows interaction of multiple biomolecules with, for example, a particular cell, thereby increasing the effect of the biomolecule. This is in contrast biomolecules bound to rigid scaffolds such as microbeads, where steric rigidity prevents multiple contact points being formed.

In certain embodiments, the second portion of the nanofilament further comprises a plurality of a first biomolecule, each biomolecule attached to a functional repeat unit of the second portion, wherein the first biomolecule is selected from the group consisting of: growth factors, cytokines, cell surface receptors (e.g. MHC complexes, T cell receptors), co-stimulatory factors (e.g. CD28, CD3), inhibitory factors (e.g. PD-1, PD-L1, CTLA4, TIM3, TIGIT), integrins, selectins, their associated natural binding partners and antibody molecules specific for said biomolecules.

In certain embodiments, the first biomolecule is a cytokine. Cytokines are soluble immune factors important for signalling to and between cells of the immune system, and include interleukins, chemokines, interferons and TNF-family molecules.

In certain embodiments, the cytokine is a pro-inflammatory cytokine, such as IL-2, IL-12, IL-7, IL-17, lL-6, IL-1, lFNα, IFNy, TNF (such as TNFα), CXCL8, CCL2, CCL3, CCL4, CCL5, CCL11, CXCL10, CCL19, CCL20, CCL21 , and GM-CSF. In certain embodiments, the cytokine is selected from IL-2, IL-7, IL-12, and lFNα. In certain preferred embodiments, the cytokine is IL-2 or lFNα.

In certain alternative embodiments, the cytokine is an anti-inflammatory cytokine. In certain such embodiments, the cytokine is selected from IL-4, TGFβ, IL-13 and IL-10.

In certain embodiments, the first biomolecule is a binding molecule. As used herein, “binding molecule” refers to molecules capable of binding to another molecule or complex. Accordingly, a binding molecule can be an antibody or binding-fragment thereof (e.g. a Fab fragment or scFv fragment), an aptamer, a ligand or a receptor, or a complex such as an MHC-antigen complex capable of binding to a T cell receptor (TCR).

In certain preferred embodiments, the binding molecule binds to a molecule present on an immune cell, for example on a T cell (e.g. a CD4+ or CD8+ T cell), B cell, dendritic cell, NK cell, NKT cell, macrophage or monocyte.

In certain embodiments the binding molecule is selected from an antibody or binding-fragment thereof (e.g. a Fab fragment or scFv fragment), an aptamer (including protein and nucleotide aptamers), a ligand or a receptor, a protein complex (for example an MHC-antigen complex capable of binding to a TCR heterodimer), and carbohydrate ligands.

In certain preferred embodiments, the binding molecule is an antibody or binding fragment thereof. Binding fragments of antibodies are known to the skilled person and include Fab, F(ab′)₂, scFv, VH or VL domains, diabodies and monovalent IgG molecules. In certain preferred embodiments the binding molecule is an antibody.

Suitable molecules presented by immune cells that may be bound by the binding molecule include TCR components or CD28 (for targeting T cells), BCR components or CD20 (for targeting B cells), CD40 (for targeting antigen presenting cells such as macrophages and dendritic cells), and CD25 (for targeting Treg cells). Suitable molecules presented by targeted immune cells would be familiar to the skilled person, and a binding molecule which binds said suitable molecule can then be selected.

In certain embodiments the biomolecule is an anti-CD3 antibody. In certain embodiments the biomolecule is an anti-CD28 antibody.

In certain embodiments, the binding molecule is selected from those binding a molecule selected from: a TCR component (e.g. CD3, the TCR heterodimer), an MHC-antigen complex, a BCR component, CD20, CD28, CD40, CD27, CD25, 4-1 BB, OX40, CD150, PD-1, CTLA4, TIM3, GARP and BTLA.

In certain such embodiments the biomolecule is attached to the second portion at a density in the range of from one molecule about each 190 nm to one molecule about each 40 nm. In certain preferred embodiments, the cytokine molecules are attached at a density of one molecule about each 130 nm or about each 90 nm.

In certain embodiments where the biomolecule is a cytokine, the biomolecule is attached to the second portion of the nanofilament at a density of at least one molecule about each 190 nm - that is, two attached biomolecules are separated by no more than about 190 nm.

In certain embodiments where the biomolecule is a cytokine, the nanofilament has from 2-40 molecules of the biomolecule attached to the second portion. In certain embodiments, the nanofilament has from 2-20 molecules of the biomolecule attached to the second portion. In certain preferred embodiments the nanofilament has from 2-10 molecules of the biomolecule attached to the second portion. In certain embodiments, the nanofilament has 2, 4 or 10 molecules of the biomolecule attached to the second portion. In certain preferred embodiments, the nanofilament has 4 molecules of the biomolecule attached to the second portion.

In certain embodiments where the biomolecule is a binding molecule, the binding molecule is attached to the second portion at a density of at least one molecule about each 130 nm -that is, two binding molecules (e.g. antibodies) are separated by no more than about 130 nm. In certain embodiments the binding molecule is attached at a density in the range of from one molecule about each 130 nm to one molecule about each 10 nm. In certain embodiments the binding molecule is attached at a density in the range of from one molecule about each 130 nm to one molecule about each 80 nm.

In certain embodiments, the nanofilament has from 2-10 molecules of the binding molecule attached to the second portion. In certain embodiments, the nanofilament has 2, 4 or 10 molecules of the binding molecule attached to the second portion. In certain embodiments, the nanofilament has 3-5 molecules of the binding molecule attached to the second portion. In certain preferred embodiments, the nanofilament has 4 molecules of the binding molecule attached to the second portion.

It will be appreciated by the skilled person that polymerisation is a random process, as is the attachment of molecules to the nanofilament. Therefore, when many nanofilaments are being produced, the above-described densities and number of molecules refer to the mean density or mean number of the molecules and do not exclude the possibility of two individual biomolecules falling outside the stated density or a particular nanofilament having a number of molecules attached that is outside the stated amount.

In certain embodiments, a plurality of a second biomolecule may be attached to the second portion of the nanofilament. In such embodiments, the second biomolecule may be independently selected from any of the embodiments described for the first biomolecule.

In such embodiments, the densities and numbers of molecules attached to the second portion described above in relation to the first biomolecule apply equally and independently to the second biomolecule.

For example, in certain embodiments, the nanofilament may comprise a plurality of molecules of a cytokine (e.g. IL-2 or IFNα) and may comprise a plurality of molecules of a second cytokine. In such embodiments, the second cytokine is a different cytokine, for example IL-2, IL-12, IL-7, IL-17, IL-6, IL-1, IFNα, IFN_{Y}, TNF (such as TNFα), CXCL8, CCL2, CCL3, CCL4, CCL5, CCL11, CXCL10, CCL19, CCL20, CCL21, and GM-CSF.

By way of further example, in certain such embodiments the nanofilament may comprise a plurality of molecules of a cytokine (e.g. IL-2 or IFNα) and may further comprise a plurality of binding molecules (e.g. antibodies). In such embodiments, the second biomolecule may be a binding molecule effective for targeting a particular cell type (e.g. a CD3 antibody for targeting T cells), where that cell type is then brought into contact with the first biomolecule (e.g. a pro-inflammatory cytokine).

As well as targeting the nanofilament to a particular subset of immune cells, the binding molecule can provide additional immunomodulatory signals to the immune cell that complement the signal provided by the attached cytokine.

For example, if a pro-inflammatory cytokine (e.g. IL-2 or IFNα) is attached, the binding molecule may be a pro-inflammatory molecule. Suitable binding molecules include those that bind a TCR component (e.g. CD3, the TCR heterodimer), an MHC-antigen complex, a BCR component, CD20, CD27, CD28, CD40, 4-1 BB or OX40. The skilled person will appreciate that to provide a pro-inflammatory signal, the binding molecule will induce signalling mediated by these molecules. Alternatively, the binding molecule may be pro-inflammatory by blocking signalling through anti-inflammatory receptors. For example, the binding molecule may block signalling through one or more of PD-1, CTLA4, TIM3, GARP or BTLA.

By way of further example, if an anti-inflammatory cytokine (e.g. TGFβ, IL-10) is attached to the nanofilament, the binding molecule may be an anti-inflammatory molecule. Suitable binding molecules include those that bind PD-1, CTLA4, TIM3, GARP or BTLA. The skilled person will appreciate that to provide an anti-inflammatory signal, the binding molecule will induce signalling mediated by these molecules. Alternatively, the binding molecule may be anti-inflammatory by blocking signalling through pro-inflammatory receptors. For example, the binding molecule may block signalling through one or more of a TCR component (e.g. CD3, the TCR heterodimer), an MHC-antigen complex, a BCR component, CD20, CD27, CD28, CD40, 4-1BB or OX40.

Accordingly, in certain embodiments, the binding molecule is pro-inflammatory. In certain such embodiments, the binding molecule binds a molecule selected from: a TCR component (e.g. CD3, the TCR heterodimer), an MHC-antigen complex, a BCR component, CD20, CD27, CD28, CD40, 4-1 BB or OX40. In certain alternative embodiments, the binding molecule binds a molecule selected from PD-1, CTLA4, TIM3, GARP or BTLA.

In certain alternative embodiments, the binding molecule is anti-inflammatory. In certain such embodiments, the binding molecule binds a molecule selected from: PD-1, CTLA4, TIM3, GARP or BTLA. In certain alternative embodiments, the binding molecule binds a molecule selected from a TCR component (e.g. CD3, the TCR heterodimer), an MHC-antigen complex, a BCR component, CD20, CD27, CD28, CD40, 4-1 BB or OX40.

In certain preferred embodiments, the binding molecule binds a molecule presented by a T cell. The provided nanofilaments are particularly effective at activating T cells when the nanofilament comprises a binding molecule capable not only of targeting T cells as a subset, but also providing a stimulatory activation signal. Without wishing to be bound by theory, this is hypothesised to be because the dynamic range of the nanofilaments allows the formation of an effective immunological synapse including interaction of both the binding molecule and cytokine with the respective receptors. As a consequence, effective multiple activation signals are delivered to the T cell, resulting in effective activation.

Accordingly, in certain preferred embodiments, the binding molecule binds a TCR component molecule. As would be familiar to the skilled person, the T cell receptor (TCR) is a complex of component molecules. These component molecules include CD3 and the TCR heterodimer, which is predominantly formed of α and β TCR chains, though may be formed of y and δ chains. In certain preferred embodiments, the binding molecule binds CD3 or a TCR heterodimer. In a preferred embodiment, the binding molecule binds CD3. In certain preferred embodiments, the second portion of the nanofilament has a plurality of binding molecules attached, where the binding molecule is an anti-CD3 antibody.

Binding of a TCR component molecule, such as CD3, is hypothesised to be particularly effective as it promotes binding of the attached cytokine by T cells, and is also thought to promote clustering of TCR components together with cytokine receptors, thereby augmenting any immune modulation (e.g. stimulatory) effect if a second biomolecule such as a cytokine is also attached to the nanofilament.

In certain embodiments, the first biomolecule is an anti-CD3 antibody and the second biomolecule is an anti-CD28 antibody.

It will also be possible to modify this CD3-binding approach, which leads to synergistic activation of T cells generally, to a targeted delivery of cytokines to antigen-specific T cells. By replacing αCD3 binding molecules with antigen-MHC complexes, the nanofilaments will deliver attached cytokines preferentially to antigen-specific T cells.

Therefore, in certain embodiments, the binding molecule is an MHC-antigen complex. Such a protein complex is able to bind a TCR heterodimer on a T cell specific for the antigen complexed with MHC (major histocompatibility complex). The particular antigen bound to the MHC complex can be any suitable T cell antigen (i.e. an antigen recognised by a T cell receptor) and could be selected by the skilled person depending on the T cell population targeted by the nanofilaments. For example, if it is intended to activate and/or expand a population of T cells for cancer therapy, the antigen forming the MHC-antigen complex attached to the nanofilament would be an antigen from the cancer to be treated. It is within the ability of the skilled person to select a suitable antigen from a targeted disease or condition.

### Methods for Producing a Nanofilament

Also provided are methods of making a nanofilament for a polymer brush. The following methods may be used to make any of the nanofilaments of the invention. Accordingly, the above-described embodiments and preferred embodiments of the nanofilaments are also embodiments and preferred embodiments of the following methods for making said nanofilaments.

According to a further aspect there is provided a method of producing a nanofilament for a polymer brush, the method comprising either:
(a)
   (i) providing a first polymerisable composition comprising an amount of a first monomer having a first functional group and an amount of a catalyst and optionally an amount of a non-functional monomer;
   (ii) polymerizing the first monomer, and if present the non-functional monomer, to provide a first polymer composition;
   (iii) adding an amount of a second monomer having a second functional group, and optionally an amount of a non-functional monomer, to the first polymer composition to provide a second polymerisable composition; and
   (iv) polymerizing the second monomer, and if present the non-functional monomer, to produce the nanofilament; or
(b)
   (i) providing a first polymerisable composition comprising an amount of a second monomer having a second functional group and an amount of a catalyst and optionally an amount of a non-functional monomer;
   (ii) polymerizing the second monomer, and if present the non-functional monomer, to provide a first polymer composition;
   (iii) adding an amount of a first monomer having a first functional group, and optionally an amount of a non-functional monomer, to the first polymer composition to provide a second polymerisable composition; and
   (iv) polymerizing the first monomer, and if present non-functional monomer, to produce the nanofilament;
wherein the first and second functional groups are different.

Preferably the method of this further aspect is suitable for producing the nanofilament of the first aspect.

In the above-described methods, the polymerisation steps (a)(ii) and (b)(iv) produce the first portion of a nanofilament according to the invention. The polymerisation steps (a)(iv) and (b)(ii) produce the second portion of a nanofilament according to the invention.

The method comprises a first step of providing a first polymerisable composition comprising an amount of a monomer having a functional group and an amount of a catalyst. The skilled person would appreciate how to select a catalyst suitable for polymerizing a known monomer.

The method further comprises a step of polymerizing the first polymerisable composition to provide a first polymer composition. The step of polymerising may be initiated by any suitable means known for the selected monomer.

The method further comprises adding an amount of another monomer having a different functional group to the first polymer composition to provide a second polymerisable composition.

The method further comprises a step of polymerising the second polymerisable composition to produce the nanofilament.

The first and second functional groups of the first and second monomers are different.

It will be understood that the polymer chains of the first polymer composition are used as a macro-initiator for polymerisation of the second polymerisable composition. Accordingly the nanofilaments produced are block copolymers comprising a first block comprising repeat units derived from the first monomer and a second block comprising repeat units derived from the second monomer.

Preferably the first and second polymerisable compositions each further comprise a plurality of non-functional monomers. Where present, the non-functional monomers of the first and second polymerisable compositions may be the same or different, preferably they are the same. Accordingly, preferably the produced nanofilaments are block copolymers comprising a first block of a first random copolymer of a first functional monomer and a non-functional monomer and second block of a second random copolymer of a second functional monomer and a non-functional monomer.

The method produces a nanofilament comprising a first portion comprising a plurality of functional repeat units each having a first functional group and optionally a plurality of non-functional repeat units. The first portion of the nanofilament is at a first end of the nanofilament and is for binding the nanofilament to a surface. Accordingly, the first functional group is for binding to a surface to be functionalised.

The produced nanofilament further comprises a second portion comprising a plurality of functional repeat units each having a second functional group and optionally a plurality of non-functional repeat units. The second portion preferably makes up the majority of the nanofilament. The second portion is intended to be ‘decorated’ with molecules such as biomolecules to provide the desired functionalisation. Accordingly, the second functional group is for binding to molecules to be bound to the nanofilaments.

By functional monomer it is meant that the monomer includes a functional group suitable for binding to another entity such as a surface or molecule. That is, a functional monomer is a monomer having a functional moiety/functional group suitable for use in an attachment reaction, such as a so-called click reaction. Suitable functional moieties will be familiar to the skilled person and include those commonly used in so-called click chemistry, such as those described in the below.

The first and second functional monomer of the method correspond to the first and second functional repeat units (respectively) of the nanofilaments according to the first aspect. That is, the first functional monomers ultimately allow for grafting the produced nanofilament to a surface to form a polymer brush. The second functional monomers are ultimately for attaching biomolecules to the second portion of the produced nanofilaments. The first and second functional groups are therefore different so that they can be selectively reacted to achieve the desired binding.

Preferably the first and second functional groups are not different by virtue of the presence of a protecting group but are distinct chemical groups.

In preferred embodiments the first and second functional groups are different groups selected from azide, N-hydroxysuccinimide (NHS), vinyl sulfone, allyl, alkynyl (e.g. acetylene, DBCO and BCN), NH₂, COOH, maleimide, tetrazine, transcyclooctene (TCO), thiol, and alkoxy. These groups have shown utility in binding to surfaces and molecules to be attached to the polymer by known methods such as so-called click chemistry.

In a preferred embodiment, the first functional group is azide. Alternatively, preferably, the first functional group is allyl.

In a preferred embodiment, the second functional group is azide. Alternatively, preferably, the second functional group is allyl.

By non-functional repeat unit it is meant that the repeat unit does not include such a functional moiety/ functional group. Primarily, the non-functional repeat units serve as spacers to separate the functional repeat units and increase the overall length of the nanofilament without including excess functional moieties.

The non-functional repeat units in the first and second portions may be the same or different. In some embodiments the non-functional repeat units are the same in both the first and second portions. Advantageously this may provide benefits in respect of synthetic simplicity.

In some preferred embodiments the polymerisable composition comprising the second monomer with a second functional group may further comprise a third monomer comprising a third functional group, wherein the third functional group is different to the first and second functional groups. The above teaching regarding the first and second functional groups applies equally to the third functional group where present. Advantageously the inclusion of a plurality of further functional repeat units in the second portion may allow the selective binding of different molecules to the nanofilament if desired.

Preferably the nanofilament of the present invention is water soluble. While it is preferred that the nanofilaments of the invention are water soluble they may also be soluble in organic solvents and may be polymerised in organic solvents.

In certain preferred embodiments the nanofilament is a polisocyanopeptide (PIC) and the polymerisation steps occur in an organic solvent. That is, the first and second polymerisable compositions may comprise an organic solvent, preferably toluene, dichloromethane or chloroform, more preferably toluene.

In a preferred embodiment the method produces a nanofilament that is a polisocyanopeptide (PIC). In certain embodiments, the PIC nanofilaments can be prepared from isocyanide monomers that have been derived from amino acids or peptides. Techniques for converting amino acids into isocyanopeptides suitable for use as monomers in the PIC polymers are known in the art.

When an isocyanopeptide monomer is derived from an amino acid, it may be derived from any suitable naturally-occurring or non-naturally-occurring amino acid, and may for example be a D- or L- amino acid (or may have an R-confirmation or an S-confirmation, referring to the chiral alpha carbon). Selection of a suitable isomer would be familiar to the skilled person and may be determined, for example, by the intended “handedness” of the PIC polymer, which is helical in structure.

In certain preferred embodiments, either the first or second functional monomer comprises an azide functional group. It is particularly preferred that the second functional monomer has an azide group because it means biomolecules can be easily conjugated to the polymer using the strain promoted azide-alkyne cyclo-addition (SPAAC) reaction.

In certain preferred embodiments, either the first or second functional monomer comprises an allyl functional group. It is particularly preferred that the first functional group is an allyl group because the allyl is small, easy to introduce into the monomer and shows excellent reactivity with tetrazoles in the nitrile imine-mediated tetrazole-ene cycloaddition (NITEC), allowing for binding to surfaces and molecules even under biological conditions.

In certain preferred embodiments:
(a) the first functional group is allyl and the second functional group is azide, or
(b) the first functional group is azide and the second functional group is allyl.

In certain embodiments the first and/or second functional group is attached the respective functional repeat units via a spacer. A spacer is a moiety covalently attaching the functional group to the backbone of the nanofilament. Advantageously, conjugation of a functional group via a spacer can provide flexibility that may be tuned for optimizing functional interactions.

In certain preferred embodiments where the first functional repeat unit comprises a spacer, the spacer is a PEG spacer. In certain such embodiments, the PEG spacer comprises from 1-10 ethylene glycol units. In certain preferred embodiments, the PEG spacer comprises from 1-5 ethylene glycol units. In certain preferred embodiments, the PEG spacer comprises 1, 2, 3, 4 or 5 ethylene glycol units. In certain preferred embodiments, the PEG spacer comprises 3 ethylene glycol units.

In certain preferred embodiments where the second functional repeat unit comprises a spacer, the spacer is a PEG spacer. In certain such embodiments, the PEG spacer comprises from 1-10 ethylene glycol units. In certain preferred embodiments, the PEG spacer comprises from 1-5 ethylene glycol units. In certain preferred embodiments, the PEG spacer comprises 1, 2, 3, 4 or 5 ethylene glycol units. In certain preferred embodiments, the PEG spacer comprises 3 ethylene glycol units.

In certain embodiments the first and/or second functional monomers used in the method have the formula:

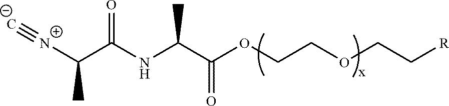

wherein R is the functional group, wherein R is different for the first functional monomer and the second functional monomer, and
wherein x is from 1 to 10, preferably x is 3-10. In a preferred embodiment, x is 3.

In certain embodiments, the first polymerisable composition comprises a plurality of non-functional monomers. In such embodiments, the non-functional monomers do not possess a reactive end group. In certain such embodiments, the non-functional monomers comprise a methoxy end group.

In certain embodiments, the second polymerisable composition comprises a plurality of non-functional monomers. In such embodiments, the non-functional monomers do not possess a reactive end group. In certain such embodiments, the non-functional monomers comprise a methoxy end group.

In those embodiments where either or both the first and second polymerisable composition comprises a non-functional monomer, the non-functional monomers preferably have the formula:

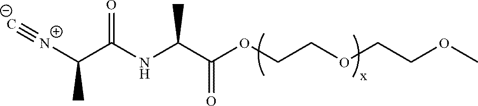

wherein x is from 1 to 10, preferably x is 3-10. In a preferred embodiment, x is 3.

It has been found that when the nanofilament comprises a PEG spacer, the length of the PEG spacer may alter the water solubility of the polymer/nanofilament. Accordingly, for water soluble nanofilaments longer PEG spacers may be preferred, for example PEG spacers comprising at least 3 glycol units. In preferred embodiments, the PEG spacer comprises from 3-10 glycol units, preferably 3-5 glycol units, more preferably 3 or 4 glycol units, most preferably 3 glycol units. These preferred embodiments apply equally and independently to the functional and non-functional repeat units.

It is demonstrated herein that multiple functional groups in the first portion of the nanofilaments produced according to the method of the invention enhances the grafting to a surface. In particular, it is shown that the produced nanofilaments show improved binding to surfaces due to the number and density of the functional groups in the first portion of the nanofilament.

Thus, in certain embodiments where a polymerisable composition comprises the first functional monomer and a non-functional monomer, the ratio of non-functional to functional monomers is from 50:1 to 1:20, such that the ratio of non-functional to functional repeat units in the first portion of nanofilaments produced according to the method is from 50:1 to 1:20. In certain embodiments, the ratio of non-functional to functional monomers is from 20:1 to 1:10, optionally 10:1 to 1:10. In certain preferred embodiments the ratio of non-functional to functional monomers is from 1:1 to 1:10. In certain preferred embodiments, the ratio of non-functional to functional repeat units is selected from 1:1, 1:3, 1:5, 1:9 and 1:10.

Thus, in certain embodiments where a polymerisable composition comprises the second functional monomer and a non-functional monomer, the ratio of non-functional to second functional monomers is from 100:1 to 10:1, such that the ratio of non-functional to functional repeat units in the second portion of nanofilaments produced according to the method is from 100:1 to 10:1. In certain preferred embodiments the ratio of non-functional to second functional monomers is from preferably from 50:1 to 10:1, preferably from 35:1 to 25:1. In preferred embodiments, the ratio of non-functional to second functional monomers is 30:1.

Preferably the method comprises the steps of: (a)
(i) providing a first polymerisable composition comprising an amount of a first monomer having a first functional group and an amount of a catalyst and optionally an amount of a non-functional monomer;
(ii) polymerizing the first monomer, and if present the non-functional monomer, to provide a first polymer composition;
(iii) adding an amount of a second monomer having a second functional group, and optionally an amount of a non-functional monomer, to the first polymer composition to provide a second polymerisable composition; and
(iv) polymerizing the second monomer, and if present the non-functional monomer, to produce the nanofilament;

That is, preferably the first portion, the portion for binding to the surface, is formed first. Advantageously, this may improve the efficiency of the method. It has been found that growing polymer chains may deactivate preventing further polymerisation of that chain. It has been found that this may be more likely when the reaction time is increased and may be more likely to occur between the first and second polymerisation steps. This may prevent the second polymerization step in either (a) or (b). Accordingly, it is preferred to form the first portion first as this is shorter and therefore has a shorter reaction time reducing the risk of deactivation.

Methods according to the invention may further comprise one or more steps for attaching the nanofilaments to a surface as below described.

Methods of the invention may further comprise one or more steps for attaching a biomolecule to the nanofilaments, as below described.

### Methods of Attaching to a Surface

In a further aspect of the invention is provided a method for producing a polymer brush, the method comprising binding to a surface a plurality of nanofilaments according to the invention. In preferred embodiments, the nanofilament is produced according to a method according to the invention.

Suitable surfaces will be appreciated by the skilled person. In certain preferred embodiments, the surface is a glass surface. In certain alternative preferred embodiments, the surface is a polymer surface. In certain embodiments, the surface is selected from a poly(methyl methacrylate) (PMMA), polystyrene (PS) or poly(lactic-co-glycolic acid (PLGA) surface.

In certain embodiments, a nanofilament of the present invention may be bound to a surface using reaction conditions under which the first functional group reacts and the second functional group does not react. Such attachment reaction results in the nanofilament being attached via the first portion, with the functional groups of the second portion free to be decorated with biomolecules using reaction conditions under which the first functional group does not react but the second functional group does react.

In certain embodiments, the nanofilament is attached to a surface before the nanofilament is decorated with biomolecules.

In certain alternative embodiments, the nanofilament has been decorated with biomolecules prior to attachment to the surface. Such embodiments are particularly preferred for flat surfaces or surfaces with a low curvature such as microbeads.

Methods for attaching biomolecules to nanofilaments according to the invention are provided below.

Suitable attachment reactions for binding the nanofilament to the surface will depend on which group is used as the first functional group, and could be selected by the skilled person accordingly. Examples include “click-chemistry” reaction pairs and streptavidin-biotin binding.

It will be appreciated by the skilled person that the surface to which the nanofilaments are to be attached is suitably functionalised for the attachment reaction.

In certain embodiments where the first functional group is azide, attachment may be via a SPAAC reaction with, for example, a DBCO (dibenzocyclooctyne-amine) group.

In certain embodiments, attachment of the nanofilament to the surface may be indirect. Such indirect attachment may be via a linker, for example. In such embodiments, the linker attaching the nanofilament to the surface is a linker on the “surface side” of the functional group of the monomer/repeat unit. The linker is distinct from any spacer that may be present as part of the functional monomer/repeat unit, though the spacer and linker may combine in order to advantageously tune the properties of the attachment, such as flexibility.

The use of such a linker can allow for a broader range of attachment reactions, thereby widening the range of surfaces to which the nanofilaments can be attached.

Thus, in certain embodiments of all aspects of the invention, the nanofilament is attached to a surface via a linker. In certain embodiments, the linker is a PEG linker, comprising 1-10 ethylene glycol units, for example 1-8 ethylene glycol units. In certain embodiments, the linker is a PEG linker comprising 1, 2, 3, 4, 5, 6, 7, 8, 9, or 10 ethylene glycol units. In certain embodiments, the linker is a PEG linker comprising 4 ethylene glycol units. In certain embodiments, the linker is a PEG linker comprising 8 ethylene glycol units

In certain embodiments where the first functional group is an azide, the nanofilament is attached to the surface via a linker comprising a SPAAC reaction partner. In certain such embodiments, attachment is via a linker comprising a DBCO group.

In certain embodiments where the first functional group is an allyl, attachment is via a linker comprising a NITEC reaction partner. In certain such embodiments, attachment is via a linker comprising a tetrazole group.

It is particularly advantageous to use allyl as the first functional group because the allyl is small and easy to introduce in the monomer. Furthermore, it is stable under polymerization conditions, as opposed to trans-cyclooctene (TCO) or tetrazine click handles that react with the nickel or isocyanides respectively. Accordingly it is particularly preferred that TCO and tetrazine handles are not used as the first functional group when the nanofilament is a PIC nanofilament.

When using allyl as the first functional group, it is further advantageous to attach the nanofilament to the surface via a linker comprising a tetrazole group. Allyl groups show excellent reactivity with tetrazoles in the nitrile imine-mediated tetrazole-ene cycloaddition (NITEC) reaction, making them particular effective for attachment. Thus, in certain embodiments the first functional group of the nanofilament is an allyl group and the nanofilament is attached to the surface by reaction with a tetrazole group.

In certain preferred such embodiments, the tetrazole group is on a linker. The NITEC reaction requires UV activation of the tetrazoles, which may denature the nanofilament. By using a linker, the UV-activation of the tetrazole can be performed before contacting the linker with the nanofilament.

Thus, in certain preferred embodiments, attachment of the nanofilament to a surface comprises the steps of first activating a linker comprising a tetrazole group, then contacting the activated linker with a nanofilament where the first functional group is an allyl, thereby attaching the linker to the nanofilament. The nanofilament-linker complex is then attached to a surface, for example by streptavidin-biotin binding, where the linker comprises one of streptavidin or biotin.

UV irradiation of PIC polymers can cause structural loss or partial destruction of the polymers. This can be avoided by first irradiating the tetrazole-containing component and subsequently combining the activated tetrazole and the PIC to perform the binding reaction.

Accordingly, in certain embodiments where a PIC polymer having allyl functional groups is used with the NITEC reaction, the UV irradiation of the tetrazole is preferably performed in the absence of the PIC polymer.

### Methods of Attaching Biomolecules

The nanofilaments of the invention and produced according to methods of the invention comprise a second portion comprising second functional groups. These second functional groups are particularly suitable for attaching biomolecules to the nanofilaments.

In certain embodiments, a nanofilament of the present invention may be decorated with biomolecules using reaction conditions under which the second functional group reacts and the first functional group does not react. Such an attachment reaction results in the functional groups of the second portion being decorated with biomolecules, but with the first functional groups of the first portion being free to attach the nanofilament to a surface.

In certain preferred embodiments, the nanofilament is attached to a surface before the nanofilament is decorated with biomolecules. In certain preferred alternative embodiments, the second portion is decorated with biomolecules before the first portion is attached to a surface. It is particularly preferred to decorate the second portion with biomolecules before attaching the nanofilament to the surface when the surface is flat or has a low curvature, such as when the surface is on microbeads.

Suitable reactions for attaching biomolecules to the second functional group of the second portion will depend on the nature of the second functional group. For example, in certain embodiments where the second functional group is an azide, the biomolecules may be attached using a SPAAC reaction.

In certain embodiments, the biomolecule may first be functionalised with a suitable reaction partner for the second functional group. In certain such embodiments where the second functional group is an azide, the biomolecules may be functionalised with a suitable SPAAC reaction partner. In certain such embodiments, the biomolecule may be functionalised with a DBCO group.

### Supports and Scaffolds

In a further aspect is provided a support comprising a surface to which a plurality of nanofilaments according to the invention is attached. In preferred embodiments, the nanofilaments attached to the surface form a polymer brush.

A support or scaffold (the terms are used interchangeably herein) according to the present invention is a substrate comprising a surface to which the nanofilaments are bound. Examples of suitable supports are microbeads, nanoparticles, glass surfaces, laboratory culture plastics and surgical implants.

In certain embodiments, the support comprises a glass surface. In certain embodiments, the support comprises a polymer surface, for example a poly(methyl methacrylate) (PMMA), polystyrene (PS) or poly(lactic-co-glycolic acid (PLGA) surface.

In certain embodiments, the support is in the form of a plurality of microbeads. In certain alternative embodiments, the support is in the form of a plurality of nanoparticles. Preferably, in such embodiments, each of the microbeads or nanoparticles comprises a surface to which a plurality of nanofilaments has been attached.

Microbeads or nanoparticles according to this embodiment of the invention may be any suitable shape, for example spherical, cylindrical, conical, tubular, cuboid, pyramidal or irregular polygonal shapes. Preferably they are substantially spherical.

As described in GB1801902.6 (which is incorporated herein by reference) nanofilaments decorated with biomolecules can be particularly effective at modulating the activity of immune cells. Supports according to the invention with nanofilaments attached will thus be particularly useful in the manipulation of immune cell activity. This presents advantages for in vitro cell culture, where supports according to the invention may be employed in laboratory techniques to modulate immune responses in culture, for example by expanding and/or activating cell populations, as demonstrated in the accompanying Examples.

Accordingly, in certain embodiments the support according to the invention is for in vitro use.

It is also advantageous to be able modulate immune cell activity in vivo. Implanting a support comprising nanofilaments decorated with biomolecules will allow modification of the immune cell response, for example by activating a particular immune cell subset or promoting antigen presentation.

Accordingly, in certain embodiments, the support is suitable for in vivo use. In certain embodiments the support is suitable for implantation in a subject. A support is suitable for implantation if it is biocompatible and does not elicit undesirable local or systemic effects once implanted in a subject. Suitable supports to which nanofilaments may be attached for in vivo use include scaffolds and matrices for tissue engineering.

The advantages of supports according to the invention in modulating the activity of immune cells compared to supports known in the art is demonstrated in the accompanying Examples. In particular, supports functionalised with nanofilaments according to the invention decorated with T cell-activating antibodies are capable of inducing a greater level of T cell activation (indicated by increased proliferation and cytokine production) compared to a comparable support to which the antibodies are directly attached (FIG. 7). Further, compared to commercially available T cell activation beads, supports functionalised with nanofilaments according to the invention induce greater cytokine production (FIG. 8). Even where the supports functionalised with nanofilaments according to the invention achieve results comparable with commercial products, the results are achieved at lower antibody concentrations, thereby reducing the material and costs required to achieve comparable effects.

Thus, in certain preferred embodiments is provided a support comprising a surface to which nanofilaments according to the invention are bound, wherein the nanofilaments are each attached to a plurality of biomolecules as described elsewhere herein.

In certain preferred embodiments, the biomolecule is selected from the group consisting of: growth factors, cytokines, cell surface receptors (e.g. MHC complexes, T cell receptors), co-stimulatory factors (e.g. CD28, CD3), inhibitory factors (e.g. PD-1, PD-L1, CTLA4, TIM3, TIGIT), integrins, selectins, their associated natural binding partners and antibody molecules specific for said biomolecules, as described above.

In certain embodiments, the nanofilaments bound to the support are each decorated with the same biomolecule.

In certain alternative embodiments, a plurality of subsets of nanofilaments of the invention is bound to the surface of the support, wherein a first subset of nanofilaments is made up of nanofilaments each of which has a plurality of a first biomolecule attached, and a second subset of nanofilaments is made up of nanofilaments each of which has a plurality of a second biomolecule. In such embodiments where the support is in the form of a plurality of microbeads or nanoparticles, nanofilaments of the first subset and nanofilaments of the second subset may be bound to the same bead or nanoparticle or may be bound to separate beads or nanoparticles.

In certain embodiments, the first biomolecule attached to the first subset of nanofilaments is a first T cell-activating molecule (e.g. antibody) and the second biomolecule attached to the second subset of nanofilaments is a second T cell-activating molecule (e.g. antibody). Preferably the first T cell-activating molecule is an anti-CD28 antibody. Preferably the second T cell-activating molecule is an anti-CD3 antibody.

In certain preferred embodiments, the support comprises nanofilaments to which anti-CD28 antibodies are attached and nanofilaments to which anti-CD3 antibodies are attached. Preferably the ratio of anti-CD28:anti-CD3 attached to the nanofilaments of the support is sufficient to activate T cells incubated with the support. Preferably the anti-CD28:anti-CD3 ratio is in the range of 6:1 to 1:2, preferably 4:1 to 1:1. In preferred embodiments the anti-CD28:anti-CD3 ratio is 4:1.

When the support includes nanofilaments decorated with one or more biomolecules, the nanofilaments and decorating biomolecules are present at a density sufficient for the biomolecules to provide their effect. For example, when the support comprises nanofilaments decorated with T cell-activating molecules, the T cell-activating molecules are present on the support at a sufficient density to activate a population of T cells incubated with the support. Suitable densities would be able to be determined by the skilled person. Suitable (non-limiting) densities of anti-CD3 and anti-CD28 antibodies for T cell activation are provided in the accompanying Examples.

In certain preferred embodiments where the support comprises a plurality of beads and comprises nanofilaments decorated with anti-CD28 antibodies and nanofilaments decorated with anti-CD3 antibodies, the antibodies are provided at a density of at least 0.5 ng per 10⁶ beads. Preferably the antibodies are provided at a density of at least 1 ng per 10⁶ beads, preferably at least 1.5 ng per 10⁶ beads, preferably at least 5 ng per 10⁶ beads, preferably at least 10 ng per 10⁶ beads.

In certain preferred embodiments where the support comprises a plurality of beads and comprises nanofilaments decorated with anti-CD28 antibodies and nanofilaments decorated with anti-CD3 antibodies, the antibodies are provided at a density in the range of from 0.5 ng per 10⁶ beads to 200 ng per 10⁶ beads. Preferably the antibodies are provided at a density in the range of from 1 ng per 10⁶ beads to 200 ng per 10⁶ beads, preferably in the range of from 1 ng per 10⁶ beads to 100 ng per 10⁶ beads, preferably in the range of from 5 ng per 10⁶ beads to 100 ng per 10⁶ beads, preferably in the range of from 10 ng per 10⁶ beads to 100 ng per 10⁶ beads.

The present invention will now be described further with reference to the following non-limiting examples.

### EXAMPLES

### Materials, Instrumentation and General Procedures

Unless stated otherwise, materials were obtained from Sigma-Aldrich (Merck) and used as obtained. Solvents were obtained from Fischer Scientific. Methoxy and azide monomers were synthesized according to the procedures in literature. Streptavidin beads MyOne C1 were bought from ThermoFischer Scientific. Streptavidin nanoparticles were obtained from Ademtech. DBCO-PEG4-NHS was obtained from JenaBioscience. Click-iT DIBO-AlexaFluor 647 was obtained from ThermoFischer Scientific. N-hydroxysuccinimide (NHS) ester of Atto488 (NHS-Atto488) was obtained from Atto-Tec. Streptavidin was bought from Sigma-Aldrich. For reactions of PIC with beads or NP, Non-Stick microfuge tubes from Ambion were used to reduce non-specific binding of PIC. Washing of the magnetic microbeads and nanoparticles was done using a magnetic rack from Westburg BV for magnetic separation. Thin layer chromatography (TLC) was performed on glass Silica gel 60 F254 plates from Merck. Flash chromatography was performed with SiliCycle silica gel 60 Å (40-63 µm). ¹H and ¹³C NMR spectra were recorded with either Bruker Avance 500 or Bruker Avance 400 NMR spectrometers. Electrospray LC-MS analysis was performed using a Finnigan LCQ Advantage lonTrap mass spectrometer. Infrared (IR) spectra were recorded on a Tensor 2700 spectrometer in ATR mode. Fluorescence was measured on a Spark M10 fluorescent plate reader using flat black 96 well plates form Corning. UV-vis measurements were performed on a Jasco V630 spectrometer. Flow cytometry was done using a BD Facs Verse cytometer and data was analyzed using FlowJo software. Confocal microscopy was performed on a Leica SP8 confocal microscope. STORM nanoscopy was performed on a low-drift inverted microscope setup

### Synthesis of PIC1-PIC5

General procedure: The desired monomers were dissolved in dry toluene obtained from MBraun SPS 800 solvent system (50 mg/mL), followed by addition of the desired catalyst solution with a catalyst to monomer ratio of 1:2000. The type of monomers and catalyst used per polymer type is given below. Polymerization was carried out overnight at room temperature. Isocyanide depletion was confirmed by disappearance of the corresponding peak by IR-spectroscopy (2140 cm⁻¹). The polymers were precipitated three times in isopropyl ether and air dried overnight. Polymers were obtained as an off-white solid. The helical backbone of the PIC was confirmed by CD-spectrometry of PIC solutions in PBS and relative polymer length was determined by viscometry (Mandal et al(2013) Chem. Sci. 4, 4168, incorporated herein by reference). For PIC5b the length was also determined by atomic force spectroscopy (AFM) as described in literature (Hammink, R. et al. (2017) Bioconjug. Chem. 28, 2560-2568, incorporated herein by reference) using a Nanoscope IV instrument (Bruker) and NSG-10 tapping mode tips (NT-MDT).

Synthesis of PIC1a-c. For polymers PIC1 a-c, different nickel carbene complexes were used as catalyst that were prepared by reacting tetrakis(isocyanide)nickel(II) perchlorate (Ni(CNR)₄(CIO4)₂) with an amine compound as described in literature (Lim, E. et al(2008) Macromolecules 41, 1945-1951, and Asaoka, S, et al(2013) ACS Macro Lett. 2, 906-911, each incorporated herein by reference). The amine compounds used for the initiation are listed below for each polymer type. Tetrakis(isocyanide)nickel(II) perchlorate was synthesized according to literature (Stephany, R. W., and Drenth, W. (1972) Recl. des Trav. Chim. des Pays-Bas 91, 1453-1458, incorporated herein by reference). Formation of the desired catalyst complexes was confirmed from the shift of the isocyanide stretching band in attenuated total reflection infrared spectroscopy (given per polymer below). The obtained catalyst solutions were then used without further purification. For the polymerizations a monomer solution of methoxy monomer (10 mg, 0.028 mmol, 40 mg/mL) was used.

PIC1a: 2-azido-1-propaneamine was used as the amine for synthesis of the catalyst (N≡C stretch: 2210 cm⁻¹). 7.33 mg (73%) was obtained.

PIC1b: azido-PEG₁₁-amine was used as the amine for catalyst synthesis (N≡C stretch: 2224 cm⁻¹). 7.41 mg (74%) was obtained.

PIC1c: Biotin-PEG8-amine was used as the amine for catalyst synthesis (N≡C stretch: 2195 cm⁻¹). 6.83 mg (68%) was obtained

Synthesis of PIC3. A monomer solution of methoxy monomer (20 mg, 0.056 mmol) was used. A solution of Ni(CIO₄)₂.6H₂O in toluene: ethanol (9:1) (1 mM) was used as catalyst solution. 15.6 mg (78%) of was obtained.

Synthesis of PIC4. A monomer solution of azide monomer (0.66 mg, 0.0018 mmol) and methoxy monomer (19.34 mg, 0.054 mmol) was used. A solution of Ni(CIO₄)₂.6H₂O in toluene: ethanol (9:1) (1 mM) was used as catalyst solution. 15.2 mg (75%) of PIC4 was obtained.

Synthesis of PIC5f. A monomer solution of allyl monomer (0.509 mg, 0.0013 mmol), azide monomer (0.632 mg, 0.0017 mmol) and methoxy monomer (18.86 mg, 0,052 mmol) was used. A solution of Ni(CIO₄)₂.6H₂O in 9:1 toluene: ethanol (1 mM) was used as catalyst solution. 16.0 mg (80%) of PIC5f was obtained.

Synthesis of PIC2 and PIC5a-e. For these polymers a small block with a monomer to catalyst ratio of 100:1 was first polymerized using a monomer solution with a final monomer concentration of 10 mg/mL and a 4 mM solution of Ni(CIO₄)₂.6H₂O in toluene:ethanol (9:1) as the catalyst solution. The composition of the monomer solution for the first block is listed per polymer below. This first block was allowed to polymerize for 10 minutes after which the appropriate amount of the solution was used as the catalyst solution for the polymerization of the second block. The polymerization time of 10 minutes is based on monomer depletion measured by a tetrazine based colorimetric assay described below. The monomer solution for the second block of PIC2 contained methoxy monomer (20 mg, 0.056 mmol). The monomer solutions for the second blocks of PIC5a-e contained azide monomer (0.66 mg, 0.0018 mmol) and methoxy monomer (19.34 mg, 0.054 mmol).

PIC2: A solution of azide monomer was used for the first block. 15.24 mg (76%) of PIC2 was obtained.

PIC5a: a solution of allyl monomer was used for the first block. 14.87 mg (89%) of PIC5a was obtained.

PIC5b: a solution of allyl monomer and methoxy monomer in a 1:1 ratio was used for the first block. 15.35 mg (77%) of PIC5b was obtained.

PIC5c: a solution of allyl monomer and methoxy monomer in a 1:3 ratio was used for the first block. 14.99 mg (75%) of PIC5c was obtained.

PIC5d: a solution of allyl monomer and methoxy monomer in a 1:9 ratio was used for the first block. 13.84 mg (69%) of PIC5d was obtained.

PIC5e: a solution of methoxy monomer was used for the first block. 14.45 mg (72%) of PIC5e was obtained.

### Tetrazine-Based Colometric Assay to Determine Polymerization Time

A polymerization with total volume 1 mL was performed using methoxy monomer at a final concentration of 10 mg/mL and 1 mM Ni(CIO₄)₂.6H₂O in toluene: ethanol (9:1) as catalyst solution with a 1:100 catalyst to monomer ratio as described above. At different time points, 10 µL of the polymerization mixture was taken out (0.278 µmol monomer) and diluted into 90 µL of a 1 mg/mL solution of benzylamino tetrazine toluene (0.48 µmol, 1,73 eq). The isocyanides left that are not polymerized at this time point react fast with the excess tetrazine. The decrease in tetrazine signal was measured with UV-vis spectroscopy at 520 nm and from this the amount of isocyanide left at the corresponding time point of the polymerization was calculated. All isocyanide was depleted after 10 minutes.

### Binding Assay with PIC1-4

Synthesis of DBCO-beads. 100 µL stock solution of streptavidin beads (MyOne C1 Streptavidin, ThermoFischer Scientific) was suspended in 100 µL borate buffer (50 mM, pH 8.5) and 1 µL of a 100 mM solution of NHS-PEG₄-DBCO in DMSO was added to react with the lysines of streptavidin. The beads were reacted overnight at room temperature and then washed 5 times with 200 µL PBS and suspended in 100 µL PBS before use. DBCO functionality on the beads was verified by reacting them with 3-hydroxy-7-azidocoumarin and measuring fluorescence by flow cytometry.

Binding assay of PIC1-4 with streptavidin and DBCO beads. PIC1 a-c, PIC3, PIC4 and PIC4-biotin were dissolved in PBS at a concentration of 0.5 mg/mL. Reactions with the beads were carried out by adding 10 µL of the polymer stock solution to 20 µL of a 10 mg/mL suspension of the corresponding beads. Control samples were made by adding 10 µL of the polymer stock solution to 20 µL PBS. All samples were left rolling overnight at room temperature. The samples were then diluted with 90 µL PBS, placed on a magnet for separation and 100 µL of the supernatant was taken out to measure the amount of polymer by circular dichroism (peak at 272 nm). The amount of binding was determined by comparing bead samples with the non-binding control. Average binding +/- standard deviation of three independent experiments was determined for each condition.

### Synthesis of Allyl Monomer

The allyl monomer was synthesized using a protocol similar to that of the methoxy monomer described in literature (Mandal et al(2013) Chem. Sci. 4, 4168, incorporated herein by reference), but instead of starting with tetraethylene glycol monomethyl ether, synthesis was started with tetraethylene glycol mono allyl ether.

Synthesis of tetra-ethylene glycol mono allyl ether. 60% sodium hydride in mineral oil (0.60 g, 14.9 mmol, 1 eq) was dissolved in pentane (1 mL) on ice. The sodium hydride solution was then slowly added to a solution of tetraethylene glycol (56.25 g, 290 mmol, 19.4 eq) in THF (50 mL) on ice. After addition was complete, a cold solution of allyl bromide (1.81 g, 14.9 mmol, 1 eq) in THF (2 mL) was slowly added and the reaction mixture was stirred on ice for 30 min at room temperature for 4 h. The reaction mixture was washed with water (3x 50 mL) and brine. The organic fraction was dried over anhydrous sodium sulfate, filtered and solvents were removed. The crude product was purified using column chromatography using ethyl acetate as eluent. The product was obtained as a colorless oil (2.65 g, 11.31 mmol, 76%). ¹H NMR (400 MHz, CDCl₃): δ = 5.90 (m, 1H), 5.27 (dm, 1H, J = 16 Hz), 5.18 (dm, 1H, J = 12 Hz), 4.02 (m, 2H), 3.75-3.57 (m, 14H). MS (ESI) m/z: found 257.4 (M+Na⁺), calculated 257.29.

Further synthesis of allyl monomer. For the rest of the synthesis of the monomer the above mentioned protocol from literature was used. In short, the ester with Boc-L-Alanine was prepared using dimethylaminopyridine (DMAP) and dicyclohexylcarbodiimide (DCC) as coupling agents. After boc-deprotection with 4 M HCl in dioxane, a peptide coupling with Boc-D-Alanine was performed using DCC and N-hydroxybenzotriazole (HOBt) as coupling agents. After deprotection the free amine was formylated with ethyl formate and then dehydrated to the isocyanide using Burgess reagent. The final product was obtained as slightly yellow oil (0.316 g, 0.818 mmol, 34% over 4 steps). ¹ H NMR (400 MHz, CDCl₃): δ = 6.99 (bd, 1H), 5.90 (m, 1 H), 5.26 (dm, 1H, J = 16 Hz), 5.17 (dm, 1H, J = 12 Hz), 4.58 (m, 1H), 4.32 (m, 2H), 4.20 (m, 1H), 4.01 (m, 2H), 3.73-3.53 (m, 14H), 1.64 (d, J = 8 Hz, 3H), 1.47 (d, J = 8 Hz, 3H). ¹³C NMR (500 MHz, CDCl₃): δ = 171.98, 165.77, 134.71, 117.12, 72.22, 70.57, 69.40, 69.13, 68.82, 64.68, 63.54, 53.43, 48.58, 19.67, 18.01 MS (ESI) m/z: found 409.4 (M+Na⁺), calculated 409.45

### Synthesis of Tetrazoles

Synthesis of Tetrazole-PEG8-biotin. Tetrazole-PEG8-biotin was synthesized by a peptide coupling of biotin-PEG8-NH2 (Click Chemistry Tools) with 4-(2-Phenyl-2H-tetrazol-5-yl)benzoic Acid (Tetrazole-COOH). Tetrazole-COOH was synthesized according to literature.³²To a solution of the tetrazole-COOH (3.89 mg, 0.0146 mmol, 1 eq) in DMF (0.5 mL) was added DIPEA (7.6 µL, 3 eq), HOBt (5.9 mg, 0.044 mmol, 3 eq), DIPCDI (5.5 mg, 0.044 mmol, 3 eq) and biotin-PEG8-amine (10 mg, 0.0146 mmol, 1 eq). The reaction was stirred at room temperature for 4h. Dichloromethane was added (2 mL) and the reaction mixture was washed 3x with 10% aqueous citric acid solution, 1x with water and 1x with brine. The organic fraction was dried over anhydrous sodium sulfate, filtered and solvents were removed. The crude product was purified by column chromatography over silica using ethyl acetate as eluent. Tetrazole-PEG8-biotin was obtained as an orange oil (11.7 mg, 0.0126 mmol, 86%). ¹H-NMR (400 MHz, CDCl₃): δ = 8.33 (m, 2H), 8.21 (m, 2H), 8.02 (m, 2H), 7.62-7.58 (m, 3H), 4.50 (m, 1H), 4.33 (m, 1H), 3.71-3.54 (m, 40H), 2.92 (dd, 1H, J=8, 4 Hz), 2.72 (d, 1H, 16 Hz), 2.22 (m, 2H), 1.46 (m, 4H), 1.31 (m, 2H). Mass (ESI) m/z: found 932.4 (M+H⁺), calculated: 932.5.

Synthesis of tetrazole-PEG₄-methyltetrazine for STORM samples. Tetrazole-PEG₄-methyltetrazine was prepared according to the protocol above using methyltetrazine-PEG₄-Amine (Click Chemistry Tools). Tetrazole-PEG₄-methyltetrazine was obtained as a pink solid (10.55 mg, 17.2 µmol, 65%). ¹H-NMR (400 MHz, CDCl₃): δ = 8.46 (m, 2H), 8.30 (m, 2H), 8.17 (m, 2H), 7.98 (m, 2H), 7.60-7.49 (m, 3H), 7.06 (bs, 1H), 7.02 (m, 2H), 4.17 (m, 2H), 3.86 (m, 2H), 3.76-3.86 (m, 12H), 3.03 (s, 3H). Mass (ESI) m/z: found:634.6 (M+H⁺), calculated: 634.7.

### STORM Nanoscopy

Sample preparation. PIC5b was reacted with tetrazole-PEG₄-methyltetrazine using a similar protocol as described for the biotinylation below. The tetrazine functional PIC5b was diluted to 1 mg/mL in PBS and 100 µL of this solution was first reacted 4h with 1 mM DBCO-biotin (Jena Bioscience) in DMSO (1 µL, 1 nmol, 0.11 eq to azides on polymer), and then with 10 mM DIBO-AlexaFluor 647 in DMSO (20 nmol, 2.27 eq to azides on polymer) for 2h. After this time 3.33 mM TCO-AlexaFluor 488 (see below) was added (6 µL, 20 nmol) and the polymer conjugation reaction were left overnight. Without further purification the dual-labeled polymers were diluted to 10 µg/mL in PBS and 20 µL of the dilution was spotted on streptavidin coated coverslips (#1.5 Micro Coverglass, Electron Microscopy Sciences, preparation described below). After a minute incubation the coverslips were thoroughly rinsed with PBS to remove unbound polymer and unreacted dyes. The coverslip sample was mounted in a custom made low-drift magnetic sample holder. Polymers were imaged in 1 mL of OxEA buffer. Imaging was performed in Wide Field illumination mode. During data acquisition, sample plane excitation power densities of approx. 1.8-5.0 kW/cm² were used for the 639 nm and 488 nm light sources respectively. Optionally, for back pumping purposes, simultaneous excitation with the 405 nm light source was used at gradually increasing excitation power densities up to maximum 0.05 kW/cm² in the sample plane. Typically, 20,000-50,000 frames were acquired in a ROI of 300x300 pixels (for each channel) with a pixel size of 0.111 µm at an exposure time of 10 ms.

### Data Analysis and Image Reconstruction

Data sets were analyzed with Fiji Image J 1.52 g³⁴ and the analysis module ThunderSTORM.³⁵ A detection threshold of 200 photons was used, typical Uncertainty Mode values were 12 nm for the 639 channel and 18 nm for the 488 channel. Images were reconstructed using the Averaged shifted histograms method, with a rendering pixel size of 10 nm. Software drift correction (ThunderSTORM) was applied using Fiducial Markers (100 nm Tetraspek, Life Technologies). Images were corrected for shifts due to chromatic aberrations, and channels were aligned using Fiducial Markers (100 nm Tetraspek; Life Technologies).

Preparation of TCO-AlexaFluor 488. A 10 mM solution of DIBO-AlexaFLuor 488 (ThermoFischer Scientific) in DMSO (10 µL, 100 nmol) was reacted with a 10 mM solution of a TCO-PEG₈-azide (12 µL, 120 nmol, 1.2 eq) for 4h. To quench the presence of unreacted TCO-azide, 50 nmol of DBCO-NH₂ (JenaBioscience) was added, the solution was diluted with PBS to final AlexaFluor 488 concentration of 3.33 mM and allowed to react for 2h. The resulting TCO-AlexaFLuor 488 was used without further purification.

### Biotinylation of PIC5a-f with the NITEC Reaction

A solution of Tetrazole-PEG8-biotin in PBS (0.7 mM, 100 µL) was prepared in a 5 mL glass vial. The vial was placed under a 254 nm lamp (Camag) without cap at a distance of 10 cm and irradiated for 10 minutes. 500 µL of a 2 mg/mL solution of the desired polymer in PBS was added to the activated tetrazole solution and stirred overnight at room temperature. After reaction the samples were diluted to 1 mg/mL with PBS before use. 50 µL of the solution was further diluted to 0.5 mg/mL with PBS and fluorescence was measured on Tecan Spark M10 platereader to confirm the reaction (excitation 368 nm, emission 514 nm).

### Binding of PIC5a-f to Streptavidin Beads and Nanoparticles

Before binding the biotinylated PIC to a surface, they were first labeled with a DIBO-AlexaFluor 647 dye. To a solution of the PIC (1 mg/mL), the desired amount of the dye was added from a 10 mM stock in DMSO and reacted overnight on a roller bank at 4° C. From the solution of labeled PIC (1 mg/mL), the desired volume was added to the desired amount of streptavidin beads or NP in a 0.5 mL Eppendorf tube. The bead or NP suspension was diluted to 4*10⁷ beads/mL or 7*10¹⁰ NP/mL with PBS and the reaction was left overnight on a roller bank. The beads/NP were washed 1x with 0.05% tween (400 µL) and 4x with PBS (400 µL. PIC beads/NP were suspended in 400 µL PBS and analyzed by flow cytometry. For quantification of polymer density, a stripping assay was developed that is described below.

### Stripping Assay to Quantify PIC Density on Beads and Nanoparticles

10 µL of the PIC-bead or NP solution was transferred to a new Eppendorf tube and supernatant was removed using magnetic separation. A 2% Tween solution in MQ (400 µL) was added and the sample was placed in a 90° C. water bath for 10 minutes and then on ice for 5 minutes. 150 µL of supernatant was taken and diluted with 50 µL PBS after which fluorescence was measured on the plate-reader. Fluorescence was compared to that of a trend line of the same PIC treated under similar conditions to determine PIC concentration in the supernatant. (The calculation of polymer density on microbeads or nanoparticles can be obtained by taking the values in ug polymer per mg bead (the value of non-specific binding of PIC2 was subtracted) and dividing by polymer molecular weight (which was estimated as 2100 (statistical number of monomers/polymer) * 360.4 (molecular weight of methoxy monomer) ₌ 757 kg/mol) to obtain mol polymer/mg bead. This was multiplied by N_{A} to obtain polymer number and divided by number of beads/mg (7*10⁸ according to manufacturer), or NP/mg (2.69*10¹¹ according to manufacturer) to obtain polymers/bead. Polymer density in polymers/µm² can be calculated by multiplying with the surface per bead (3.14 µm²), or surface per NP (0.041 µm²). The spacing between polymers was calculated by taking the square root of the density.). Stripped beads were washed trice with PBS and analyzed with flow cytometry to verify that all polymer was stripped from the beads.

### BSA Conjugation to PIC Beads and Nanoparticles

Preparation of labelled BSA. BSA (0.5 mg, 7.5 nmol) was dissolved in 0.05 M borate buffer pH 8.5 (0.5 mL) and 1.5 µL of a 10 mM solution of NHS-Atto488 in DMSO was added (2 eq). The reaction was stirred for 2 hours at room temperature. 250 µL of the reaction was transferred to another Eppendorf tube and 1 µL of a 10 mM solution of NHS-PEG4-DBCO (2.67 eq) in DMSO was added and the reaction was left for 2 hours at room temperature to make the DBCO-Atto488-BSA. Both the Atto488-BSA and the DBCO-Atto488-BSA were purified over a Zeba desalting column (5 mL, ThermoFischer Scientific) using the manufacturers protocol. After purification the protein concentration and degree of labeling were determined by UV-vis spectrometry on a NanoDrop 2000 spectrometer (ThermoFischer Scientific). Samples were diluted to 0.1 mg/mL BSA before use. Obtained degree of labeling: Atto488-BSA: 1.89 Atto488/BSA. DBCO-Atto488-BSA: 1.90 Atto488/BSA, 2.2 DBCO/BSA.

BSA conjugation of beads and nanoparticles. For the SPAAC first method, 50 µL of the desired polymer solution (1 mg/mL) was diluted with PBS (100 µL) and the desired BSA solution was added (50 µL, 5 mg/mL). The reaction was left rolling overnight at room temperature. 10 µL of the resulting BSA-polymer solution was then diluted with PBS (30 µL) and the desired bead of NP stock (as obtained from supplier) was added (10 µL). The beads/NP were rolled overnight at room temperature and then washed as described above for polymer bead reactions.

For the graft first method, PIC beads and NP were prepared as described above. After washing they were suspended in PBS (40 µL) and DBCO-BSA solution was added (10 µL, 5 mg/mL). The beads were left rolling at room temperature overnight and were washed according to the protocol described above.

### Grafting of PIC to Glass Coverslips

Preparation of streptavidin coated coverslips. Glass coverslips were cleaned by sonication in aqueous 10% NaOH (5 min), followed by sonication in isopropanol (5 min) and drying under nitrogen flow. The glass was then immersed in piranha solution for 30 min, washed five times with MilliQ water and dried under nitrogen flow. The coverslips were placed in a 2% (3-Glycidyloxypropyl)trimethoxysilane solution in dry toluene for 30 min. Coverslips were washed twice with toluene and trice with ethanol and placed in an oven at 80° C. for 30 minutes. Silanized coverslips were placed overnight in a 20 µg/mL solution of streptavidin in borate buffer (50 mM, pH 8.5). Streptavidin coverslips were placed in wells of a 6-well plate (one per well) and washed with PBS (seven times 3 mL, shaking one min per wash) and used immediately. To verify the surface coating steps, contact angles were measured using a home built set up for piranha cleaned glass, GOPTMS silanized glass and streptavidin coated glass, and were found to be 11°, 50° and 26° respectively (average of three coverslips per condition).

Binding of PIC to streptavidin coated coverslips. 500 µL of a 1 mg/mL solution of the desired PIC was placed on the desired coverslip and was then left at room temperature for 4 hours. The liquid was then aspirated with a pipette and the coverslips were washed with PBS (7x 3 mL) as described above. The PIC coated coverslips were analyzed by confocal microscopy. Z-stacks of each sample were made measuring fluorescence of the Alexa647 on the polymer. For each sample some cells (peripheral blood myeloid cells) labeled with a violet dye were added and allowed to drop to the bottom before measuring to determine the z height of the coverslip surface.

### Results

### Synthesis of End Functional PIC

An aqueous ‘grafting to’ approach was chosen to synthesize the semiflexible PIC brushes to increase versatility. Brushes of related hydrophobic PIC have been prepared by growing them from the surface, but this required organic solvents. The ‘grafting to’ approach allows for polymerization in organic solvents followed by grafting under aqueous conditions which broadens applicability to surfaces that don’t withstand organic solvents, for example cultureware plastics or the surfaces of polymer particles for nanomedicine. In addition, this approach provides the possibility to functionalize and characterize the PIC in solution before surface grafting.

This ‘grafting to’ approach requires PIC with a functional group for surface binding that is only present at one end of the polymer. To establish synthetic protocols for the production of ‘end-functional’ water soluble PIC and to test the feasibility of surface immobilization of these polymers, we started with the synthesis of end-functional polymers on an otherwise unfunctionalized polymer. The first approach towards end-functional PIC relies on using a nickel catalyst that is made by initiation of a tetrakis(tert-butylisocyanide) nickel(II) complex with a nucleophile attached to a functional group.

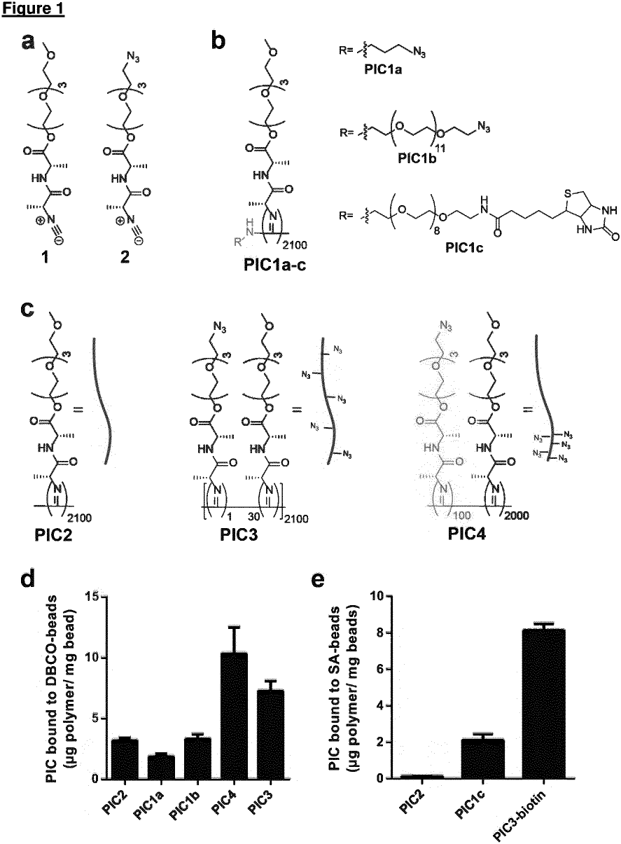

Polymerization with the initiated nickel complex yields polymers with the functional group of the nucleophile at one end of the polymer. To test this approach, we used the azide as the functional group since it is known to be compatible with polymerization of the isocyanide. We also investigated the use of biotin as an initiator, which allows for binding of the resulting polymer to one of many commercially available streptavidin functional surfaces. Three different end functional PICs were synthesized using this strategy, containing as end functional group an azidopropane (PIC1a), an azide with an ethylene glycol (PEG₁₂) spacer (PIC1b), and a biotin with an ethylene glycol (PEGs) spacer (PIC1c). For these and all polymers described in these Examples, a catalyst to monomer ratio of 1:2100 was used to yield polymers with a length of around 200 nm. Structures of polymers PIC1 a-c are shown in FIGS. 1a and 1b.

The capabilities of these three end-functional PICs to be grafted onto a surface under aqueous conditions were tested by binding them to 1 µm magnetic beads with a surface that binds their end-functional groups (DBCO-surface for PIC1a-b, streptavidin-surface for PIC1c). As a negative control, a polymer without any functional groups was synthesized (PIC2, FIG. 1c). As a positive control, a polymer with azides along the entire polymer chain was prepared by random copolymerization of azide and methoxy monomers in a ratio of 1:30 (PIC3, FIG. 1c). For binding to the streptavidin beads, the azides of PIC3 were first changed into biotin functionalities by reaction with a DBCO linked to a biotin. To determine the amount of polymer grafted onto the beads, the concentrations of PIC solutions in PBS were measured both before and after incubation with circular dichroism spectroscopy. PIC1a-c could not be efficiently grafted to surface as the amount of grafted polymers was either very low compared to positive control PIC3 or indiscernible from the negative control.

A second approach towards end-functional PIC was developed, using a small block of monomers with a functional group for surface grafting. First, a small block (catalyst to monomer ratio of 1:100) of monomers with an azide in the side chain was polymerized. These azide monomers have been previously used to add functionality to these polymers. This block was used as a macro-initiator for a longer second block (catalyst to monomer ratio of 1:2000) that contains only non-functional methoxy monomers. The polymer made using this strategy (PIC4, FIG. 1c) has multiple azides at one end for surface binding, as opposed to the polymers made by the first strategy that only have one.

When PIC4 was bound to the DBCO-beads, binding higher than the nonspecific binding of negative control polymer PIC2, and slightly higher than for positive control PIC3 was observed (FIG. 1d). The non-specific binding on these DBCO beads was higher than observed for streptavidin beads, which is attributed to the hydrophobicity of DBCO groups.

The observed binding of end-functional PIC4 of 10 µg per mg beads corresponds to a polymer density of around 2600 polymers per µm² and a spacing of around 20 nm between polymers. The fact that binding is observed for PIC4, but not for PIC1a-c, suggests that multiple functional groups are needed for efficient grafting to a bead surface.

The second strategy to synthesize end-functional PIC using a small block of multiple reactive monomers provided enhanced, more efficient grafting and was chosen for the development of PIC brushes that can be further modified using click chemistry.

### Addition of a Second Functional Group to the Polymer

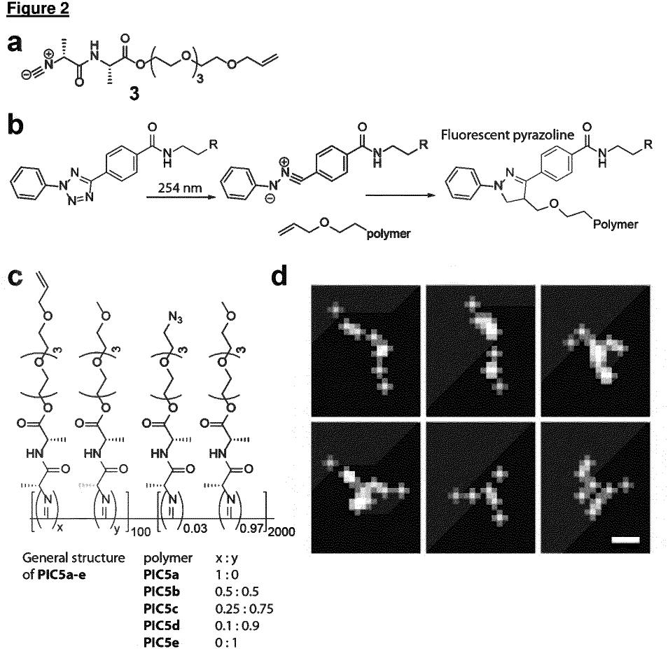

Once established that PIC with a small block of surface reactive monomers can be efficiently grafted to a surface, a different monomer with allyl functionality (FIG. 2a) was designed for use in the starting block. This allows for the use of the azide monomers in the second block for the conjugation of (bio)molecules via the SPAAC reaction. The allyl was chosen because of its compatibility with the isocyanide polymerization, as opposed to trans-cyclooctene (TCO) or tetrazine click handles that react with the nickel or isocyanides respectively. Furthermore, the allyl is easy to introduce in the monomer and shows excellent reactivity with tetrazoles in the nitrile imine-mediated tetrazole-ene cycloaddition (NITEC) reaction. In the NITEC reaction a tetrazole is activated by ultraviolet (UV) irradiation to a reactive nitrile-imine intermediate that reacts efficiently with alkenes such as allyls to form a pyrazoline product that is fluorescent (FIG. 2b).

Preferably, the UV irradiation of the NITEC reaction is not performed at the same time as the surface grafting. This way the grafting can also be applied on surfaces that are sensitive to UV and in situations where homogenous irradiation of UV light is difficult, for instance particle suspensions or surfaces with 3D structures. To achieve this, we designed a two-step conjugation protocol. First, the NITEC reaction is used to convert the allyl groups at the start of the polymer to a different reactive group. In the second step, this newly introduced group is then used to couple the PIC to the surface. Another advantage of this two-step approach is that the NITEC reaction is performed under conditions with only the allyl polymer and desired functional group present. This prevents known side reactions of the nitrile-imine intermediate with nucleophiles that may be present during grafting in presence of biomolecules.⁴

This approach also allows for the introduction of end functional groups that otherwise would not have been stable under polymerization conditions, such as tetrazines, and thereby expands the range of surface functional groups that may be used to tether the polymers.

A series of polymers was made in similar fashion as PIC4, but with a first block containing either only allyl monomers (PIC5a), allyl and methoxy monomers in a ratio of 1:1 (PIC5b), 1:3 (PIC5c), 1:9 (PIC5d) or only methoxy monomers (PIC5e) and a second block containing azide and methoxy monomers in a ratio of 30:1 (FIG. 2c). To verify that the synthesis resulted in polymers with allyl monomers present in a small block (around 10 nm) at one end of the chain and azides present in remaining chain, PIC5b was modified so that azides were functionalized with an AlexaFluor 647 dye. The allyl groups were functionalized with an AlexaFluor 488 dye. The allyl functionalization was performed by first converting the allyl to a tetrazine via the NITEC reaction with a tetrazine linked to tetrazole, followed by reaction with a TCO containing AlexaFluor 488. The resulting polymers were visualized using stochastic optical reconstruction microscopy (STORM) to confirm a two block polymer with an azide containing block of around 200 nm visible with 647 nm excitation, and a small allyl containing block of 10-30 nm visible by 488 nm excitation (FIG. 2d).

### Grafting of the Allyl Containing PIC via the Two Step Protocol

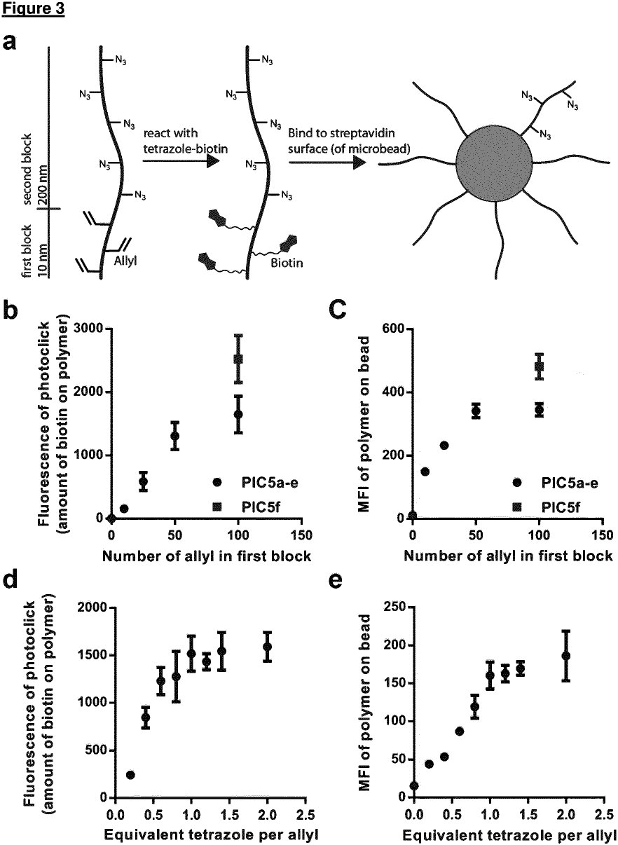

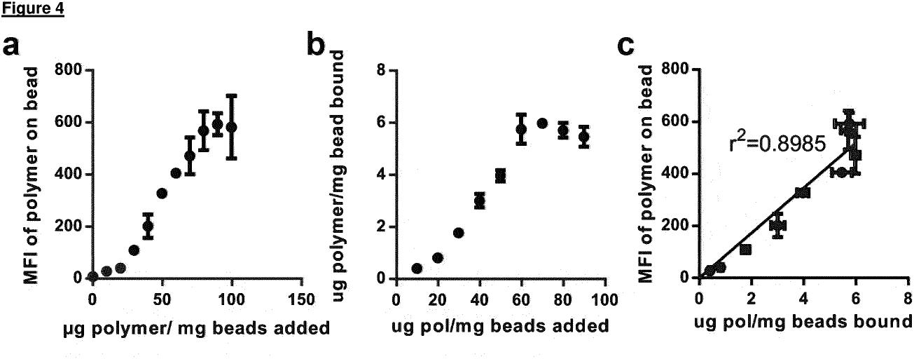

To demonstrate the feasibility of surface grafting using the above mentioned two step protocol, biotin was used as the functional group for surface grafting because it allows for grafting to the commercially available magnetic streptavidin beads which previously showed low non- specific binding of PIC (data not shown). For this purpose tetrazole was connected to biotin via a PEGs linker to increase solubility in aqueous solution (tetrazole-PEGs-biotin). In the first step, this tetrazole-PEGs-biotin can be used to conjugate biotins to the allyl groups of PIC5a-e with the NITEC reaction, which allows for surface grafting of the biotinylated PIC to streptavidin surfaces in the second step (FIG. 3a).

The biotinylation of the allyl-containing PIC was investigated using fluorescence as readout. Reaction of photo-activated tetrazole-PEGs-biotin with an aqueous solution of PIC5a gave rise to a fluorescent signal with an emission at 415 nm. In contrast, only a very weak fluorescence was observed when the tetrazole was reacted a solution of PIC3 which contains no allyl groups. Using the increase in fluorescence as a measure for biotinylation, the UV irradiation time for tetrazole activation and the reaction time with the allyl polymer were optimized to establish the final biotinylation protocol.

Using this protocol, polymers PIC5a-e were reacted with an excess of photo-activated tetrazole-biotin corresponding to 200 equivalents per polymer to keep the background fluorescence the same in all samples. The resulting fluorescence of the NITEC reaction increases with the increasing statistical amount of allyl groups present (FIG. 3b). For PIC5a with 100 allyl monomers in the first block, however, similar fluorescent intensity is observed as for PIC5b with only 50 allyl monomers. A possible explanation may be that at statistical densities of allyl monomers higher than 50 out of 100, the reaction with activated tetrazole is limited by steric hindrance. As more biotins are clicked onto the polymer, they may shield the availability of the remaining allyl groups. To test if more fluorescence is observed when the same amount of allyl groups is spread over the whole polymer to increase spacing, a new polymer was made with the same length and ratio of monomers as PIC5a, but with both the allyl and azide monomer randomly distributed along the polymer (PIC5f). The observed fluorescence after reaction of PIC5f with 200 equivalent of tetrazole-biotin is roughly twice as high as for PIC5a (red square in FIG. 3b). This observation explains why for PIC5a the observed degree of biotinylation is limited, and implies that if the 100 allyl groups are not confined to the first block, the additional spacing allows more of them to react.

Next, the biotinylated products of PIC5a-f were bound to 1 µm magnetic streptavidin beads under aqueous conditions after labeling of the polymers with a DIBO-Alexa Fluor 647 dyes in the side chain (on average two dye molecules per polymer). The amount of labeled polymer bound to the beads was quantified by flow cytometry. The mean fluorescent intensity (MFI) of the Alexa Fluor 647 signal on the beads was plotted against the number of allyl groups in the first block (FIG. 3b). The amount of polymer on the beads correlates well with the amount of biotins in the first block and no binding is observed for PIC5e with no allyl groups.

To gain more insight in the efficiency of biotin conjugation with the NITEC reaction, PIC5b was reacted with different equivalents of tetrazole-PEG₈-biotin. The fluorescence of these reactions was compared to control reactions with PIC3 that lacks the allyl groups. The fluorescence of the conjugated biotin increases with the amount of tetrazole added and reaches a plateau around one equivalent of tetrazole (FIG. 3d). This verifies that biotin conjugation with the NITEC is highly efficient with very few side reactions.

The PIC obtained from the biotinylation of PIC5b with different equivalents of tetrazole-biotin were also labeled with Alexa Fluor 647 (on average one dye molecule per polymer) and bound to beads. The surface grafting of these PIC increases with increasing amounts of biotin present in the first block (FIG. 3e). These results are in line with the previous finding that only limited binding was observed for polymers made with only one group for surface grafting and confirms the need of multiple surface binding groups for efficient grafting. Optimal biotinylation and grafting efficiency was found for PIC5b reacted with one equivalent of biotin to allyl, so these polymers (biotin-PIC5-647) were used to quantify grafting densities.

### Quantification of Grafting Densities

To determine the maximum amount of polymer that can be grafted to the beads, a series of beads was prepared by adding increasing amounts of biotin-PIC5b-647 per bead. The amount of polymer bound to the beads as measured by flow cytometry was plotted against the amount of polymer added and shows that a maximum amount of polymer is reached when around 100 µg polymer per mg bead is added (FIG. 4a). To quantify the amount of bound polymer on these beads, PIC-beads were incubated in 2% SDS at 90° C. for 10 minutes to break the biotin-streptavidin bonds and release the dye labeled polymers. The fluorescence of the supernatants was measured after magnetic separation and the amount of polymer was determined using a trend line of unconjugated biotin-PIC5b-647 in order to calculate the polymer density that was on the beads. The stripped beads were measured by flow cytometry to verify that all bound polymer had been removed.

The polymer densities obtained by this stripping assay were plotted against the amount of polymer added (FIG. 4b), and shows a similar binding curve as shown in FIG. 4a. The amount of bound polymer according to the stripping assay correlates well with the amount of polymer on the beads as measured by flow cytometry (FIG. 4c). The maximum amount of bound polymer is around 6 µg polymer/mg beads which corresponds to a density of ~2174 polymers per µm² and an average spacing of around 21.5 nm. The obtained density implies that the surface is indeed in the brush regime with a spacing smaller than two times the radius of gyration.

### Synthesis of PIC Brushes on Nanoparticles and Glass Coverslips

Having shown that PIC starting with a block of biotin can be grafted to the surface of streptavidin microbeads, next we tested the versatility of this method. To demonstrate that this approach can be extended to a wide range of surfaces we also performed surface grafting on glass coverslips and streptavidin nanoparticles (NP) with a diameter of 200 nm.

A series of PIC-conjugated nanoparticles was prepared using increasing amounts biotin-PIC5b-647 in similar fashion as described for the microbeads. For the NP the maximum observed polymer density was found to be 1600 polymers per µm² (spacing of 25 nm), which is comparable to the maximum density on the microbeads. To graft the polymers to glass coverslips, streptavidin-coated coverslips were first prepared and grafting of Biotin-PIC5b-647 to these surfaces was confirmed by confocal microscopy.

The successful grafting of the biotinylated PIC on microbeads, nanoparticles and coverslips demonstrates that this aqueous grafting strategy is widely applicable on a range of surfaces.

### Protein Functionalization of Azide Functional PIC Brushes

To show that the obtained PIC surfaces can be functionalized with biomolecules using the azides in the side chains of the polymers, PIC microbeads and NP were functionalized with Atto488 labeled bovine serum albumin (BSA) containing DBCO click handles. BSA functionalized PIC brushes can be made in two ways. One possibility is to first conjugate DBCO-BSA to the polymer via the SPAAC reaction, followed by grafting of the BSA-polymers to the surface (‘SPAAC first’). Alternatively, the polymer can be first grafted to a surface, followed by BSA functionalization of the PIC brushes (‘graft first’). PIC-NP and PIC microbeads were prepared via both methods. The labeled BSA was also added to the beads and particles lacking PIC to determine non-specific binding levels. The Atto488 fluorescence of the labeled BSA and the AlexaFlour 647 fluorescence of the labeled PIC on the beads were measured by flow cytometry.

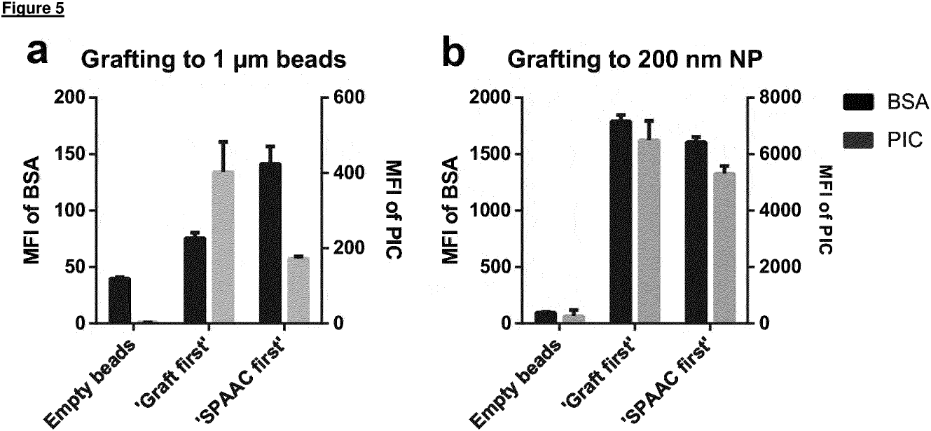

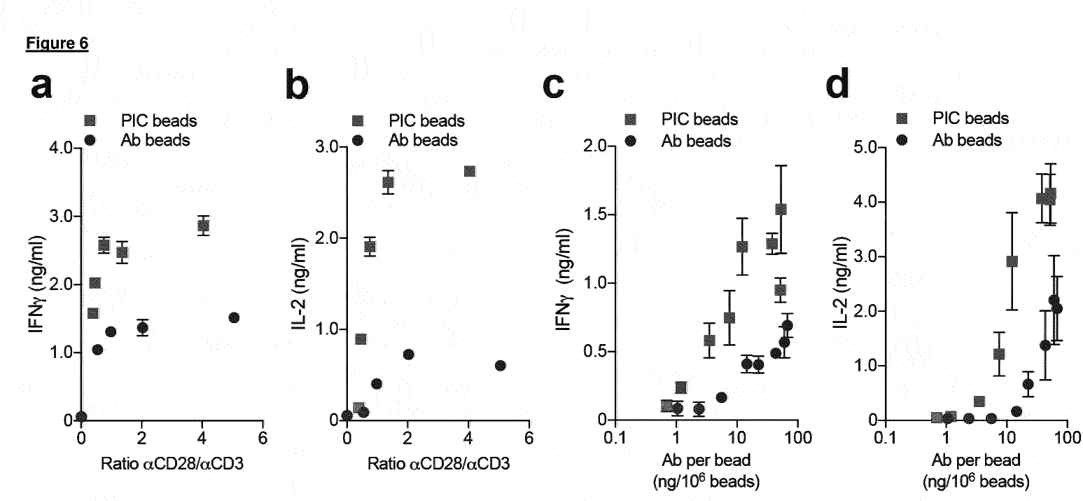

For the conjugations using microbeads it was found that the ‘graft first’ strategy results in a twofold increase of the polymer density, but a twofold decrease in BSA conjugation when compared to the ‘SPAAC first’ strategy (FIG. 5a). The BSA signal obtained with the ‘graft first’ strategy is only slightly higher than that of the nonspecifically bound BSA on empty beads, which implies that at these polymer densities BSA conjugation is limited by poor availability of the azides in the brush. In the case of the NP, both strategies yield similar biofunctional brushes (FIG. 5b).

The fact that biofunctionalization of the NP with the ‘graft first’ strategy works as well as with the ‘SPAAC first’ strategy may be explained by the higher curvature of the NP surface compared to microbeads. The free ends of the PIC grafted on the NP will therefore be more spaced than on the beads, thereby exposing more azides for biofunctionalization.

### Improved Functionality of PIC-Grafted Biomolecules

The advantages of the end-functional nanofilaments for attachment of biomolecules to surfaces were demonstrated using in a T cell activation assay.

Anti-CD3 and anti-CD28 antibodies bound to microbeads are routinely used in cell culture systems to activate T cells to divide and or produce pro-inflammatory cytokines such as IFNy and IL-2. To compare the effect of attaching the antibodies to beads using the end functional nanofilaments instead of directly to the microbead (as is conventional), PIC nanofilaments were functionalized with anti-CD3 and anti-CD28 antibodies and attached to microbeads at a ratio of 4:1 aCD28:aCD3 using the methods described herein. PIC-functionalised beads were then compared to the conventional microbeads also having a ratio of 4:1 aCD28:aCD3 antibodies.

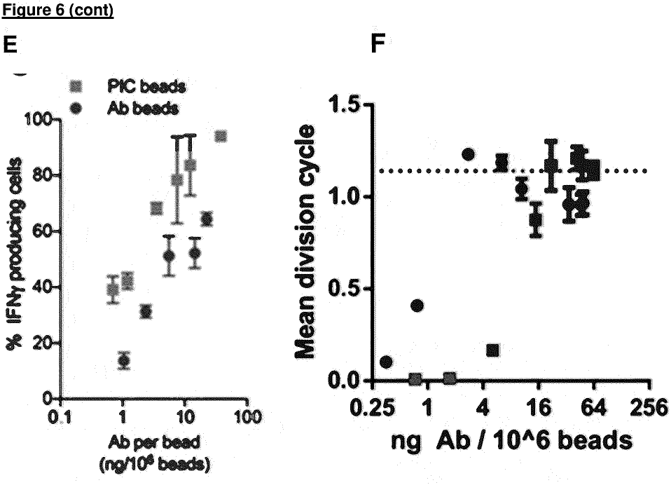

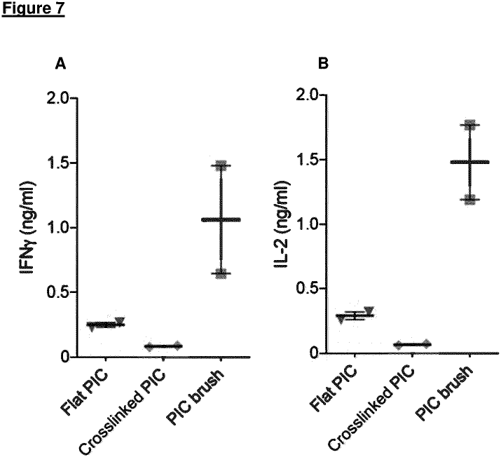

FIG. 6 demonstrates that PIC-functionalised beads (PIC-Ab beads - squares) stimulated more pro-inflammatory cytokine release compared to conventional CD3:CD28 microbeads with the antibodies directly attached. FIGS. 6A and B shows that improved cytokine levels (A: IFNy; B: IL-2) were observed for PIC-Ab beads across all ratios of aCD28 to aCD3 antibodies tested. FIGS. 6C and D show that PIC-Ab beads induce more cytokine production than direct-attachment beads at all densities of antibodies tested (ng of antibody per 10⁶ beads). PIC-Ab beads also induced a greater percentage of cultured cells to produce pro-inflammatory cytokine IFNy (FIG. 6E). In addition, PIC-Ab beads more potently induced T cell proliferation compared to direct-attachment beads (FIG. 6F).

FIG. 7 demonstrates the advantages of the block-copolymer nanofilaments provided herein when presenting biomolecules, for example when attached to a support or scaffold. FIGS. 7A and B show potent stimulation of IFNy and IL-2 production by T cells by nanofilaments decorated with aCD3 and a CD28 antibodies when attached to a scaffold such that they form a polymer brush.

In contrast, attachment of conventional random copolymer PIC nanofilaments such that the filaments are bound “flat” to the scaffold surface resulted in negligible cytokine production under the same conditions (24 hours of incubation with PIC-Ab beads (aCD3:aCD28)). Similarly, cross-linking of the block-copolymer nanofilaments such that they do not behave as a polymer brush resulted in reduced cytokine production (FIGS. 7A and B-“Crosslinked PIC”).

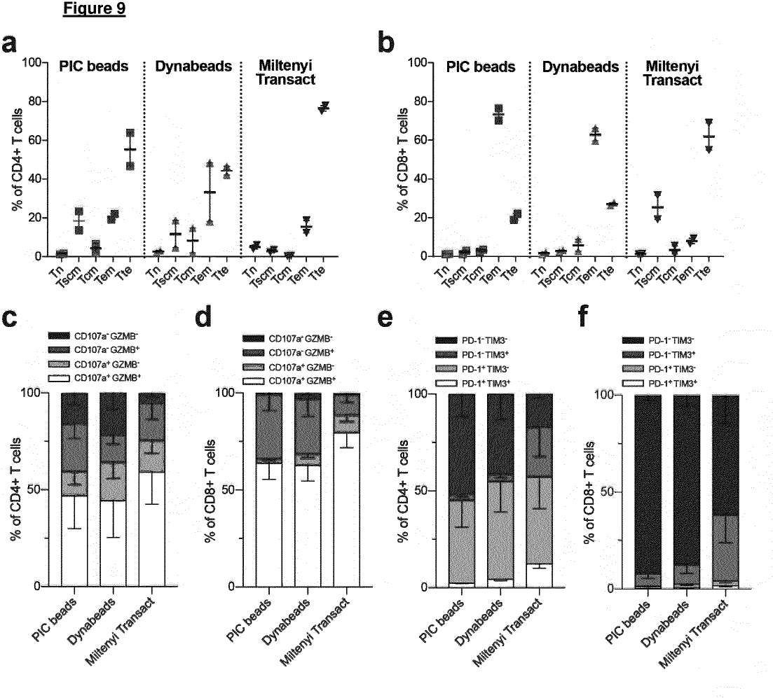

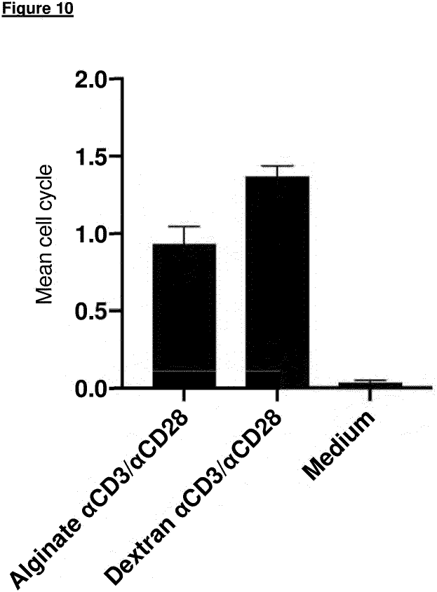

To further demonstrate the advantages of the nanofilaments provided herein, aCD3/aCD28 functionalized nanofilaments were attached to microbeads and compared with industry standard aCD3/aCD28 stimulation scaffolds (Dynabeads® and Miltenyi Transact®) in a typical and clinical T cell ex vivo expansion protocol (FIGS. 8 and 9).

FIGS. 8A and B shows T cell IFNy (A) and IL-2 (B) production measured after 24h incubation with the different scaffolds. FIG. 8C shows T cell proliferation as measured by mean division cycle after 3 days and FIG. 8D shows fold expansion of cells after 7 and 14 days of incubation with the stimulatory scaffolds. The PIC beads show more potent IFNy induction than any of the commercial scaffolds, and induce at least equivalent IL-2 production, proliferation and expansion. None of the commercial scaffolds performs as well as the PIC beads across all assays.

FIG. 9 further characterizes the T cell population following stimulation with the PIC beads or the commercial aCD3/aCD28 scaffolds. It can be seen from panels A and B that incubation with PIC beads results in CD4 and CD8 T cells characterized by a more balanced effector profile than the commercial beads, e.g. the Miltenyi Transact scaffold. This is indicated by fewer T cells in the “Tte” or “T effector” category and more in the “Tem” (“effector memory”) and/or “Tscm” (“memory stem cell”) subsets. Tem and Tscm T cells are not yet terminally differentiated effector cells (in contrast to Tte cells) and are thus less likely to become quickly immunologically “exhausted”.

FIGS. 9C and D show the expression of degranulation and cytotoxicity markers (CD107 and granzyme B) exhibited by T cells stimulated with the different scaffolds. The PIC beads are at least equivalent to one or both of the commercial scaffolds in terms of the profile of the exposed T cells.

FIG. 9E and F show the expression of regulatory markers (also known as “immune checkpoint receptors”) on T cells exposed to the scaffolds. T cells exposed to the PIC beads exhibit a greater proportion of T cells that are negative for both PD-1 and TIM3, indicating a more activated and less exhausted phenotype.

FIGS. 8 and 9 demonstrate that attachment of functionalized nanofilaments as provided herein to a support such as beads provides a more potent means for influencing the cellular targets - in this case, a more potent stimulation of T cell activation and proliferation using aCD3 and aCD28 antibodies. Put another way, an equivalent of cellular modulation (e.g. T cell activation and/or proliferation) can be induced using lower concentrations of biological material.

These data demonstrated that the end functional nanofilaments provided herein provide the means to more effectively functionalise surfaces with molecules such as biomolecules.

### Conclusion

In conclusion, we demonstrate the synthesis of an end functional semi-flexible PIC with lengths of around 200 nm. These polymers possess a small block of multiple allyl functional monomers present only at one end of the polymer and azide groups present in the rest of the polymer chain. These allyl functionalities can be efficiently reacted with tetrazoles to introduce new functional groups such as a biotin at one end of the PIC. The biotinylated PIC can be grafted to a wide range of substrates under aqueous conditions to obtain PIC brushes on surfaces of glass coverslips, 1 µm beads and 200 nm nanoparticles. On the particles and microbeads polymer brushes with densities of around 2000 polymers per µm² could be reached. The amount of polymer grafted to these surfaces increases with the amount of surface binding functionalities present at the end of the polymer. For the synthesis of bioactive brush surfaces often large biomolecules such as proteins need to be attached to the brush. We showed that this can be easily done by conjugation of the azides in the brush using DBCO functionalized BSA as a model protein. Polymer functionalization can be performed either before or after surface grafting for grafting to the surface 200 nm nanoparticles. For grafting to the surface of microbeads, BSA needs to conjugated to the PIC before surface grafting. The PIC filaments have long length, are easily functionalized and are unique in their semi-flexibility. The possibility to graft these stiff polymers to a wide range of surfaces under aqueous conditions and modify them with a variety of biological functions will be a useful addition to toolbox of polymers as bioactive surfaces. Moreover, the data presented here shows that bioactive surfaces made using the provided nanofilaments result in a more potent effect on the cellular targets. Thus, greater responses can be induced compared to current commercial products or, put another way, an equivalent of cellular modulation (e.g. T cell activation and/or proliferation) can be induced using smaller amounts of biological material.

### Non-PIC Nanofilaments

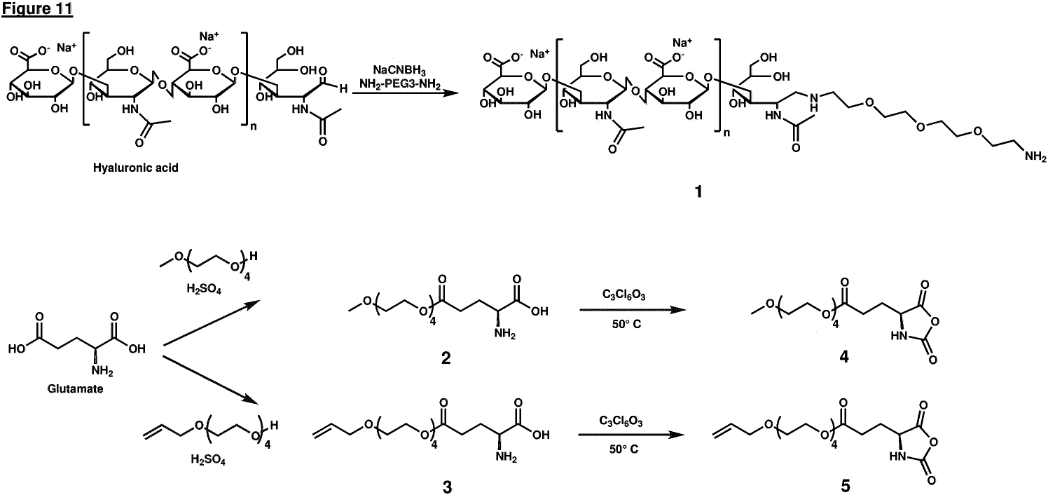

In addition to nanofilaments according to the invention formed from PIC, nanofilaments can be formed of alternative polymers. Nanofilaments formed of alginate or dextran polymers and functionalized with aCD3 and aCD28 antibodies are capable of inducing T cell proliferation, albeit when provided in solution (FIG. 10).

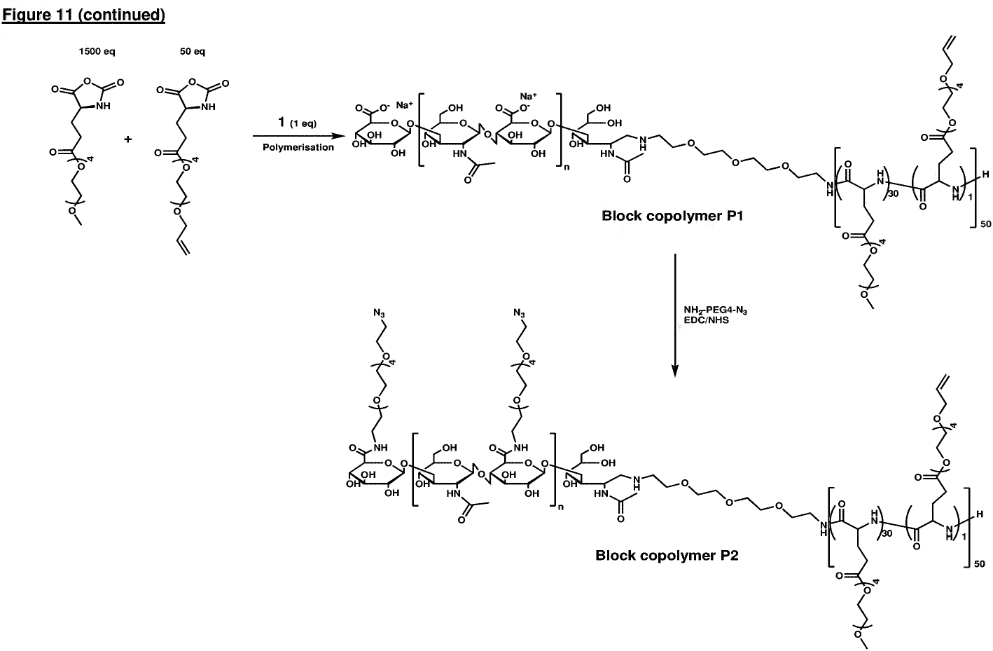

Semi-flexible nanofilament block-copolymers similar to PIC and able to be functionalized in the same way can be prepared as polysaccharide-poly glutamate polymers, e.g. hyaluronic acid-poly glutamate polymers. These block copolymers can then be attached to a surface to form a polymer brush as described above in relation to PIC nanofilaments. A suitable synthetic process for making hyaluronic acid-poly glutamate polymers block co-polymers is described below in reference to FIG. 11:

### Synthesis of Functional Polysaccharide (Hyaluronic Acid)-Glutamate Block Copolymers

Hyaluronic acid (HA) is mixed with 1 equivalent diamine PEG3 linker and is treated with sodium cyanoborohydride in a reductive amination reaction to synthesize amine functionalized HA (1).

Next glutamate is treated with monomethyl tetraethylene glycol under acidic conditions to synthesize compound 2. In a similar fashion, 3 is synthesized starting from allyl tetra ethylene glycol.

Compounds 2 and 3 are then converted to the corresponding N-Carboxyanhydrides (NCA) using triphosgene to obtain 4 and 5. These NCA monomers are mixed in a 30:1 ratio (compound 4: compound 5) and polymerized using compound 1 as the initiator. The obtained HA-glutamate block copolymer P1 is further functionalized to P2 with amine-PEG4-azide in a standard peptide coupling to convert the carboxylic acids of HA to azide handles.

The block copolymer P2 can be conjugated to biomolecules and attached to surfaces in a similar manner as described above for PIC polymers.

Although the synthesis scheme presented above and in FIG. 11 is described in relation to HA, it would be within the ability of the skilled person to adapt it to other polysaccharides so as to produce other polysaccharide-glutamate block copolymers nanofilaments according to the present invention.

1. A nanofilament for forming a polymer brush, wherein the nanofilament comprises first and second portions;
wherein the first portion comprises a plurality of functional repeat units each having a first functional group and optionally a plurality of non-functional repeat units;
wherein the second portion comprises a plurality of functional repeat units each having a second functional group and optionally a plurality of non-functional repeat units; and
wherein the first and second functional groups are different.

2. (canceled)

3. The nanofilament of claim 1, wherein the first and second functional groups are selected from azide, N-hydroxysuccinimide (NHS), vinyl sulfone, allyl, alkynyl (e.g. acetylene, DBCO and BCN), NH₂, COOH, maleimide, tetrazine, transcyclooctene (TCO), thiol and alkoxy.

4. The nanofilament of claim 1, wherein the nanofilament is a polyisocyanopeptide (PIC).

5. The nanofilament of claim 4, wherein the nanofilament comprises repeat units of the formula: 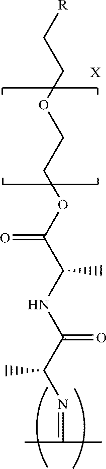
wherein R is selected from azide, N-hydroxysuccinimide (NHS), vinyl sulfone, allyl, alkynyl, NH₂, COOH , maleimide, tetrazine, transcyclooctene (TCO), thiol and alkoxy,
and wherein X is 1-10.

6. The nanofilament of claim 1, wherein the first and/or second portion comprises a polysaccharide polymer, optionally wherein the polysaccharide is selected from: hyaluronic acid, alginate, pectin, chitin, guar, carrageenan, carboxymethylcellulose, heparin, and dextran.

7. The nanofilament of claim 5, wherein the second portion is a polysaccharide polymer and the first portion is a poly-glutamate polymer, optionally wherein the nanofilament is a hyaluronic acid-poly-glutamate co-polymer.

8. (canceled)

9. The nanofilament of claim 1, wherein the nanofilament comprises from 1,000 to 20,000 repeat units, preferably from 1,000 to 10,000, preferably 2,000 to 5,000.

10. The nanofilament of claim 1, wherein the first portion comprises from 2 to 200 repeat units, preferably from 50 to 150.

11. The nanofilament of claim 1, wherein when the first portion comprises non-functional repeat units, the ratio of non-functional to functional repeat units in the first portion is from 50:1 to 1:20, preferably from 20:1 to 1:10.

12. (canceled)

13. The nanofilament of claim 1, wherein when the second portion comprises non-functional repeat units, the ratio of non-functional to functional repeat units in the second portion is from 50:1 to 10:1, preferably from 35:1 to 25:1.

14. (canceled)

15. The nanofilament of claim 1, wherein:
(a) the first functional group is allyl and the second functional group is azide, or
(b) the first functional group is azide and the second functional group is allyl.

16. The nanofilament of claim 1, further comprising a plurality of a biomolecule attached to the second portion, optionally wherein the biomolecule is selected from a cytokine and a binding molecule.

17. (canceled)

18. The nanofilament of claim 16, wherein the biomolecule is a cytokine selected from IL-2, IL-12, IL-7, IL-17, IL-6, IL-1, IFNα, IFNγ, TNF (such as TNFα), CXCL8, CCL2, CCL3, CCL4, CCL5, CCL11, CXCL10, CCL19, CCL20, CCL21, and GM-CSFor wherein the biomolecule is a binding molecule selected from an antibody or binding-fragment thereof, an aptamer, a ligand or a receptor, or an MHC-antigen complex.

19. (canceled)

20. A method of producing a nanofilament for a polymer brush, the method comprising either:
(a)
(i) providing a first polymerisable composition comprising an amount of a first monomer having a first functional group and an amount of a catalyst and optionally an amount of a second non-functional monomer;
(ii) polymerizing the first monomer, and if present the non-functional monomer, to provide a first polymer composition;
(iii) adding an amount of a second monomer having a second functional group, and optionally an amount of a non-functional monomer, to the first polymer composition to provide a second polymerisable composition; and
(iv) polymerizing the second monomer, and if present the non-functional monomer, to produce the nanofilament; or
(b)
(i) providing a first polymerisable composition comprising an amount of a second monomer having a second functional group and an amount of a catalyst and optionally an amount of a non-functional monomer;
(ii) polymerizing the first monomer, and if present the non-functional monomer, to provide a first polymer composition;
(iii) adding an amount of a first monomer having a first functional group, and optionally an amount of a non-functional monomer, to the first polymer composition to provide a second polymerisable composition; and
(iv) polymerizing the first monomer, and if present non-functional monomer, to produce the nanofilament;
wherein the first and second functional groups are different.

21. The method of claim 20, wherein the first polymerisable composition of (a) or the second polymerisable composition of (b) comprises an amount of a non-functional monomer and the molar ratio of the first monomer to the non-functional monomer is from 50:1 to 1:20, preferably from 20:1 to 1:10.

22. (canceled)

23. The method of claim 20, wherein the second polymerisable composition of (a) or the first polymerisable composition of (b) comprises an amount of a non-functional monomer and the molar ratio of the second monomer to the non-functional monomer is from 100:1 to 10:1, preferably from 50:1 to 10:1, preferably from 35:1 to 25:1, preferably 30:1.

24. The method of claim 20, wherein in (a) the molar ratio of monomer to catalyst based on the content of all monomers present in the first polymerisable composition is from 200:1 to 2:1, preferably from 150:1 to 50:1.

25. The method of claim 20, wherein the molar ratio of monomer to catalyst based on the content of all monomers present in the second composition is from 20,000:1 to 1,000:1, preferably from 10,000:1 to 1,000:1, preferably from 5,000:1 to 2,000:1.

26. The method of claim 20, further comprising attaching a plurality of biomolecules to the produced nanofilament, optionally wherein the plurality of biomolecules are attached to the nanofilament by reaction with the second functional group.

27. (canceled)

28. The method of claim 26, where the biomolecule is selected from one or more of a cytokine or a binding molecule.

29. A nanofilament obtainable by the method of claim 20 .

30. A polymer brush comprising a plurality of the nanofilaments of claim 1.

31. A method of producing a polymer brush comprising binding to a surface a plurality of nanofilaments according to claim 1.

32. (canceled)

33. The method of claim 31, wherein binding the nanofilaments to the surface comprises first binding the nanofilaments to a linker by reaction with the first functional group, then binding the linker-nanofilament conjugate to the surface, such that the nanofilaments are attached to the surface via the linker.

34. The method of claim 31, wherein when the nanofilaments do not comprise a biomolecule before binding to the surface, the method further comprises the step of attaching a plurality of biomolecules to the nanofilaments attached to the surface.

35. A scaffold comprising a surface to which a plurality of nanofilaments according to claim 1 have been bound, optionally wherein the plurality of nanofilaments form a polymer brush.

36-39. (canceled)
